SEQUENCE LISTING (34 sequences)

SEQ ID NO: 1 - DNA, 18 bp, synthetic construct
  source 1..18: mol_type = other DNA
ccatgaatcctgtggagc

SEQ ID NO: 2 - DNA, 18 bp, synthetic construct
  source 1..18: mol_type = other DNA
gtagagggtttgccgatg

SEQ ID NO: 3 - DNA, 20 bp, synthetic construct
  source 1..20: mol_type = other DNA
gtgaagccggacaaggagaa

SEQ ID NO: 4 - DNA, 20 bp, synthetic construct
  source 1..20: mol_type = other DNA
tggctgaacaccttcccaaa

SEQ ID NO: 5 - DNA, 21 bp, synthetic construct
  source 1..21: mol_type = other DNA
gaataacccgaattggagcag

SEQ ID NO: 6 - DNA, 21 bp, synthetic construct
  source 1..21: mol_type = other DNA
agcgattcctcttcacagttg

SEQ ID NO: 7 - DNA, 20 bp, synthetic construct
  source 1..20: mol_type = other DNA
agtctccaagcgacgaaaaa

SEQ ID NO: 8 - DNA, 20 bp, synthetic construct
  source 1..20: mol_type = other DNA
gcaagaagcctctccttgaa

SEQ ID NO: 9 - DNA, 19 bp, synthetic construct
  source 1..19: mol_type = other DNA
tcactggtgagaaccccct

SEQ ID NO: 10 - DNA, 19 bp, synthetic construct
  source 1..19: mol_type = other DNA
cctgattcacacggcgtag

SEQ ID NO: 11 - DNA, 20 bp, synthetic construct
  source 1..20: mol_type = other DNA
agcttgctggtgaaaaggac

SEQ ID NO: 12 - DNA, 20 bp, synthetic construct
  source 1..20: mol_type = other DNA
ttatagtcaagggcatatcc

SEQ ID NO: 13 - DNA, 20 bp, synthetic construct
  source 1..20: mol_type = other DNA
gccttggatctcttgatgga

SEQ ID NO: 14 - DNA, 20 bp, synthetic construct
  source 1..20: mol_type = other DNA
ttcttggctcttatgcgatg

SEQ ID NO: 15 - DNA, 20 bp, synthetic construct
  source 1..20: mol_type = other DNA
agggtgtggcatcctacctt

SEQ ID NO: 16 - DNA, 20 bp, synthetic construct
  source 1..20: mol_type = other DNA
ggtccatgatggcgttcact

SEQ ID NO: 17 - DNA, 20 bp, synthetic construct
  source 1..20: mol_type = other DNA
ttccaactgcatacccgacc

SEQ ID NO: 18 - DNA, 21 bp, synthetic construct
  source 1..21: mol_type = other DNA
tcgtggtctttggtaatgggg

SEQ ID NO: 19 - DNA, 20 bp, synthetic construct
  source 1..20: mol_type = other DNA
actctggcctacacactgga

SEQ ID NO: 20 - DNA, 20 bp, synthetic construct
  source 1..20: mol_type = other DNA
tgacgatgggacggagtttg

SEQ ID NO: 21 - DNA, 21 bp, synthetic construct
  source 1..21: mol_type = other DNA
ctccaacacattcctttacac

SEQ ID NO: 22 - DNA, 20 bp, synthetic construct
  source 1..20: mol_type = other DNA
actcaaccttcaaatagcct

SEQ ID NO: 23 - DNA, 21 bp, synthetic construct
  source 1..21: mol_type = other DNA
tgctgacagtcttgcaggtag

SEQ ID NO: 24 - DNA, 20 bp, synthetic construct
  source 1..20: mol_type = other DNA
tacccccacagtgccataga

SEQ ID NO: 25 - DNA, 19 bp, synthetic construct
  source 1..19: mol_type = other DNA
catccttcaccaccaagag

SEQ ID NO: 26 - DNA, 18 bp, synthetic construct
  source 1..18: mol_type = other DNA
cagattgcagaaggacgg

SEQ ID NO: 27 - DNA, 22 bp, synthetic construct
  source 1..22: mol_type = other DNA
accccaagtaccatcaagggag

SEQ ID NO: 28 - DNA, 20 bp, synthetic construct
  source 1..20: mol_type = other DNA
tcacacaggacttgggtcag

SEQ ID NO: 29 - DNA, 21 bp, synthetic construct
  source 1..21: mol_type = other DNA
ccccagaacctaggcaaacat

SEQ ID NO: 30 - DNA, 22 bp, synthetic construct
  source 1..22: mol_type = other DNA
acattggtcacaattcacggta

SEQ ID NO: 31 - DNA, 20 bp, synthetic construct
  source 1..20: mol_type = other DNA
cccaggaggacggggtataa

SEQ ID NO: 32 - DNA, 19 bp, synthetic construct
  source 1..19: mol_type = other DNA
agttttctgcagcccctgg

SEQ ID NO: 33 - DNA, 19 bp, synthetic construct
  source 1..19: mol_type = other DNA
gcatctgcatggcggtaga

SEQ ID NO: 34 - DNA, 20 bp, synthetic construct
  source 1..20: mol_type = other DNA
tgccacagctcttggaacat

(19) United States
(12) Patent Application Publication
(10) Pub. No.: US 2025/0002856 A1
(43) Pub. Date: Jan. 2, 2025

(54) CULTURE-EXPANDED CANINE PROGENITOR CELLS AND RELATED METHODS

(71) Applicant: Healios KK., Tokyo (JP)

(72) Inventors: Robert J. DEANS, Riverside, CA (US); Annelies BOGAERTS, Meldert (BE); David CRAEYE, Dendermonde (BE)

(21) Appl. No.: 18/707,451

(22) PCT Filed: Nov. 4, 2022
(86) PCT No.: PCT/US2022/049049

§ 371 Date: May 3, 2024

Related U.S. Application Data
(60) Provisional application No. 63/276,552, filed on Nov. 5, 2021.

(51) Int. Cl. C12N 5/077; A61K 35/12; C12N 5/0775

(52) U.S. Cl. CPC C12N 5/0653; A61K 35/12; C12N 5/0654; C12N 5/0665; C12N 2500/25; C12N 2500/32; C12N 2501/11; C12N 2501/115; C12N 2501/135; C12N 2501/15

(57) ABSTRACT

One embodiment of the invention provides culture-expanded canine progenitor cells of non-embryonic origin that can be maintained in culture in the undifferentiated state or differentiated to form cells of multiple tissue types. The culture-expanded cells are post-natal somatic cells that can be characterized by one or more of the following: a population doubling rate of less than about 24 hours; a normal karyotype; can differentiate into at least two cell types of the mesodermal germ layer; extended replication in culture and express markers of extended replication, such as telomerase; and express markers of pluripotency. Also provided are methods of isolation and culture as well as therapeutic uses for the cells, such as treating musculoskeletal and inflammatory diseases and disorders. Additionally provided are cell banks that can be used to provide the cells for administration to a subject, drug discovery methods, and compositions of cells, such as in pharmaceutical compositions.

### FIELD OF THE INVENTION

The invention provides culture-expanded canine progenitor cells of post-natal origin that can be maintained in culture in the undifferentiated state or differentiated to form cells of multiple tissue types. Also provided are methods of isolation and culture as well as therapeutic uses for the culture-expanded canine progenitor cells. The culture-expanded canine progenitor cells are post-natal somatic cells that are capable of extended replication in culture and can be characterized by one or more of the following through about 55 population doublings: a population doubling rate of less than about 24 hours; a normal karyotype; the ability to differentiate into at least two cell types of the mesodermal germ layer; and expression of markers of extended replication (e.g., telomerase) and markers of pluripotency (e.g., oct4). The invention is also directed to methods of treating musculoskeletal disorders and inflammatory conditions using the culture-expanded canine progenitor cells. The invention is also directed to cell banks that can be used to provide the culture-expanded canine progenitor cells for administration to a canine subject. The invention is also directed to drug discovery methods. The invention is also directed to compositions of culture-expanded canine progenitor cells, such as in pharmaceutical compositions.

### BACKGROUND

To date, stem cells have been used, mostly experimentally, for treatments of a variety of diseases in different animal species. The initial focus of regenerative veterinary medicine was directed to the orthopedic diseases, but the focus is now rapidly expanding to other areas such as orodental and digestive tract diseases, liver, renal, cardiac, respiratory, neuromuscular, dermal, olfactory, and reproductive system diseases. Stem cell treatments were most often used in dogs and horses for various diseases of various organ systems, and in cats for renal, respiratory, and inflammatory diseases.

### SUMMARY OF THE INVENTION

The inventors have found canine progenitor cells of post-natal origin that can be maintained in culture in an undifferentiated state or differentiated to form cells of multiple tissue types. The canine progenitor cells are capable of extended replication in culture and can be characterized by one or more of the following through about 55 population doublings: a population doubling rate of less than about 24 hours; a normal karyotype; the ability to differentiate into at least two cell types of the mesodermal germ layer; and expression of markers of extended replication (e.g., telomerase) and markers of pluripotency (e.g., oct4).

Based at least on these findings, the invention provides canine progenitor cells and methods including, but not limited to, culture-expanded canine progenitor cells, compositions comprising the culture-expanded canine progenitor cells, methods for treating musculoskeletal disorders and inflammatory conditions using the culture-expanded canine progenitor cells, methods for constructing a cell bank using the culture-expanded canine progenitor cells, and drug discovery methods.

Accordingly, one embodiment of the invention includes culture-expanded canine progenitor cells that have a population doubling rate of less than about 24 hours, a normal karyotype, are positive for expression of telomerase and CD90, and are negative for expression of CD45 and CD34.

In one embodiment, the invention includes a method for treating an inflammatory condition in a canine comprising administering to the canine a therapeutically effective amount of culture-expanded canine progenitor cells, the cells having a population doubling rate of less than about 24 hours, a normal karyotype, are positive for expression of telomerase and CD90, and are negative for expression of CD45 and CD34.

In one embodiment, the invention includes a method for treating an inflammatory condition in a canine comprising administering to the canine a therapeutically effective amount of culture-expanded canine progenitor cells, the cells having a population doubling rate of less than about 24 hours, a normal karyotype, are positive for expression of telomerase and CD90, are negative for expression of CD45 and CD34, and can differentiate into at least two cell types of the mesodermal germ layer.

In one embodiment, the invention includes a method for treating a musculoskeletal disorder in a canine comprising administering to the canine a therapeutically effective amount of culture-expanded canine progenitor cells, the cells having a population doubling rate of less than about 24 hours, a normal karyotype, are positive for expression of telomerase and CD90, and are negative for expression of CD45 and CD34.

In one embodiment, the invention includes a method for treating a musculoskeletal disorder in a canine comprising administering to the canine a therapeutically effective amount of culture-expanded canine progenitor cells, the cells having a population doubling rate of less than about 24 hours, a normal karyotype, are positive for expression of telomerase and CD90, are negative for expression of CD45 and CD34, and can differentiate into at least two cell types of the mesodermal germ layer.

In one example, the culture-expanded canine progenitor cells have undergone at least 40 population doublings in culture.

In another example, the culture-expanded canine progenitor cells have undergone at least 50 population doublings in culture.

In one example, the culture-expanded canine progenitor cells have a population doubling rate of about 15 to 24 hours in culture.

In another example, the culture-expanded canine progenitor cells have a population doubling rate of about 16 hours in culture.

In one example, the culture-expanded canine progenitor cells are derived from bone marrow, adipose tissue, umbilical cord blood, or placental tissue.

In one example, the culture-expanded canine progenitor cells are positive for expression of CD29.

In one example, the culture-expanded canine progenitor cells are negative for expression of MHC Class II.

In one example, the culture-expanded canine progenitor cells are positive for expression of one or more of PTHLH, CD13, CD44, CD49c, CD73, CD90, CD105, and IL1R2.

In one example, the culture-expanded canine progenitor cells are positive for expression of IL1R2.

In one example, the culture-expanded canine progenitor cells are negative for expression of rex-1, CD34, CD45, and NOV.

In one example, the culture-expanded canine progenitor cells are positive for expression of nanog, sox-2 and oct-4.

In one example, the culture-expanded canine progenitor cells express telomerase up to about 55 population doublings in culture.

In one example, the culture-expanded canine progenitor cells are capable of reducing or inhibiting T-cell proliferation in vivo and/or in vitro.

In one example, the culture-expanded canine progenitor cells are capable of providing angiogenesis in vivo and/or in vitro.

In one example, the culture-expanded canine progenitor cells are capable of differentiating into at least two cell types of the mesodermal germ layer.

In another embodiment, the culture-expanded canine progenitor cells have a population doubling rate of about 16 hours, a normal karyotype, are positive for expression of telomerase and CD90, and are negative for expression of MHC Class II, CD45 and CD34. The culture-expanded progenitor cells have undergone at least 40 population doublings in culture, are also positive for expression of one or more of PTHLH, CD13, CD44, CD49c, CD73, CD105 and IL1R2, and are derived from bone marrow.

In one example, the inflammatory condition is a chronic inflammatory condition or an acute inflammatory condition.

In one example, the acute or chronic inflammatory condition is one of dermatitis, inflammatory eye disease, inflammatory brain disease, inflammatory airway disease, and inflammatory bowel disease.

In another example, the dermatitis is atopic dermatitis.

In another example, the inflammatory eye disease is keratoconjunctivitis.

In another example, the inflammatory brain disease is meningoencephalomyelitis.

In one example, the inflammatory condition is an autoimmune disease.

In one example, the musculoskeletal disorder is one of osteoarthritis and cruciate ligament rupture.

In another example, the cruciate ligament rupture is a partial cruciate ligament rupture.

In one example, the musculoskeletal disorder is a spinal condition.

In another example, the spinal condition is one of a spinal cord injury and intervertebral disc disease.

In one embodiment, the culture-expanded canine progenitor cells include, but are not limited to, post-natal somatic cells having some characteristics of embryonic stem cells, but being derived from post-natal tissue, and providing the effects described in this application. The culture-expanded canine progenitor cells may naturally achieve these effects (i.e., not genetically or pharmaceutically modified). However, natural expressors can be genetically or pharmaceutically modified to increase potency.

Culture-expanded canine progenitor cells may express pluripotency markers, such as oct4. They may also express markers associated with extended replicative capacity, such as telomerase. Other characteristics of pluripotency can include the ability to differentiate into different cell types of the mesodermal germ layer. Such canine progenitor cells are not tumorigenic, do not form teratomas, and are not immortalized or transformed in culture. The canine progenitor cells may be highly expanded while maintaining a normal karyotype. For example, in one embodiment, the canine progenitor cells have undergone at least 10-60 cell doublings in culture, such as 10-15, 15-20, 20-25, 25-30, 30-35, 35-40, 40-45, 45-50, 50-55, 55-60, or more cell doublings, wherein the cells have a normal karyotype, express telomerase, express oct-4, and differentiate into at least two cell types of the mesodermal germ layer. Furthermore, the culture-expanded canine progenitor cells have a population doubling rate of less than about 24 hours.

The culture-expanded canine progenitor cells may be prepared by the isolation and culture conditions described herein.

The canine progenitor cells include, but are not limited to, the following numbered embodiments:

1. Culture-expanded canine progenitor cells that have a population doubling rate of less than about 24 hours, a normal karyotype, can differentiate into at least two cell types of the mesodermal germ layer, and are post-natal somatic cells.

2. Culture-expanded canine progenitor cells that have a population doubling rate of less than about 24 hours, a normal karyotype, express telomerase, and are post-natal somatic cells.

3. Culture-expanded canine progenitor cells that have a population doubling rate of less than about 24 hours, a normal karyotype, express oct-4, and are post-natal somatic cells.

4. Culture-expanded canine progenitor cells that have a population doubling rate of less than about 24 hours, a normal karyotype, have undergone at least 40 population doublings in culture, and are post-natal somatic cells.

5. The culture-expanded canine progenitor cells of any one of 1-3 above wherein the cells have undergone at least 40 population doublings in culture.

6. The culture-expanded canine progenitor cells of 5 above wherein the cells have undergone at least 50 population doublings in culture.

7. The culture-expanded canine progenitor cells of any one of 1-6 above having a population doubling rate of about 15 to 24 hours in culture.

8. The culture-expanded canine progenitor cells of any one of 1-7 above having a population doubling rate of about 16 hours in culture.

9. The culture-expanded canine progenitor cells of any one of 1-8 above being derived from bone marrow, adipose tissue, umbilical cord blood, or placental tissue.

10. The culture-expanded canine progenitor cells of any one of 1-9 above being positive for expression of CD90 and negative for expression of CD45 and CD34.

11. The culture-expanded canine progenitor cells of any one of 1-10 above being positive for expression of CD29.

12. The culture-expanded canine progenitor cells of any one of 1-11 above being negative for expression of MHC Class II.

13. The culture-expanded canine progenitor cells of any one of 1-12 above being positive for expression of one or more of PTHLH, CD13, CD44, CD49c, CD73, CD105, and IL1R2.

14. The culture-expanded canine progenitor cells of any one of 1-13 above being positive for expression of IL1R2.

15. The culture-expanded canine progenitor cells of any one of 1-14 above being negative for expression of rex-1 and NOV.

16. The culture-expanded canine progenitor cells of any one of 1-2 and 4-15 above being positive for expression of nanog, sox-2 and oct-4.

17. The culture-expanded canine progenitor cells of any one of 1-16 above expressing telomerase up to about 55 population doublings in culture.

18. The culture-expanded canine progenitor cells of any one of 1-17 above expressing oct-4 up to about 55 population doublings in culture.

19. The culture-expanded canine progenitor cells of any one of 1-18 differentiating into at least two cell types of the mesodermal germ layer up to about 55 population doublings in culture.

20. The culture-expanded canine progenitor cells of any one of 1-19 above reducing or inhibiting T-cell proliferation in vitro and/or in vivo.

21. The culture-expanded canine progenitor cells of any one of 1-20 above being capable of providing angiogenesis in vitro and/or in vivo.

22. The culture-expanded canine progenitor cells of any one of 2-21 above being capable of differentiating into at least two cell types of the mesodermal germ layer.

23. The culture-expanded canine progenitor cells of 22 above being capable of differentiating into at least two of an osteoblast, an adipocyte, and a chondrocyte.

24. The culture-expanded canine progenitor cells of any one of 1-23 above wherein the cells are not tumorigenic, do not form teratomas, are not transformed, and are not immortalized.

25. The culture-expanded progenitor cells of any one of 1-24 above prepared by a method comprising: obtaining tissue from a canine; establishing a population of adherent cells; selecting cells that positively express CD90 and/or do not express at least one of CD45 and CD34; and expanding the selected cells in a culture media.

26. A composition comprising the culture-expanded progenitor cells of any one of 1-24 above and a second component.

27. A pharmaceutical composition comprising the culture-expanded progenitor cells of any one of 1-24 above and a pharmaceutically acceptable carrier.

28. A kit comprising the following separately packaged components: the culture-expanded progenitor cells of any one of 1-24 above; culture media; and instructions for culturing the cells.

29. A method for preparing the composition of 26 above, comprising: obtaining tissue from a canine; establishing a population of adherent cells; selecting cells that positively express CD90 and/or do not express at least one of CD45 and CD34; expanding the selected cells in a culture medium; and adding the cells to the second component.

30. A method for preparing the pharmaceutical composition of 27 above, comprising admixing the culture-expanded progenitor cells with the pharmaceutically acceptable carrier.

31. A method for preparing the culture-expanded canine progenitor cells of any one of 1-24 above, the method comprising: obtaining tissue from a canine; establishing a population of adherent cells; selecting cells that positively express CD90 and/or do not express at least one of CD45 and CD34; and expanding the selected cells in a culture medium.

32. A method to construct a cell bank, the method comprising expanding and storing the culture-expanded progenitor cells of any one of 1-24 above for future administration to a subject.

33. A method for drug discovery, the method comprising exposing the culture-expanded progenitor cells of any one of 1-24 above to an agent to assess one or more effects of the agent on the cells.

34. A method for treating an inflammatory condition in a canine comprising administering to the canine a therapeutically effective amount of the canine progenitor cells of any one of 1-24 above.

35. The method of 34 above wherein the inflammatory condition is a chronic inflammatory condition or an acute inflammatory condition.

36. The method of 35 above wherein the acute or chronic inflammatory condition is one of dermatitis, inflammatory eye disease, inflammatory brain disease, inflammatory airway disease, and inflammatory bowel disease.

37. The method of 36 above wherein the dermatitis is atopic dermatitis.

38. The method of 36 above wherein the inflammatory eye disease is keratoconjunctivitis.

39. The method of 36 above wherein the inflammatory brain disease is meningoencephalomyelitis.

40. The method of 34 above wherein the inflammatory condition is an autoimmune disease.

41. A method for treating a musculoskeletal disorder in a canine comprising administering to the canine a therapeutically effective amount of the canine progenitor cells of any one of 1-24 above.

42. The method of 41 above wherein the musculoskeletal disorder is one of osteoarthritis and cruciate ligament rupture.

43. The method of 42 above wherein the cruciate ligament rupture is a partial cruciate ligament rupture.

44. The method of 42 above wherein the musculoskeletal disorder is a spinal condition.

45. The method of 44 above wherein the spinal condition is one of a spinal cord injury and intervertebral disc disease.

The canine progenitor cells include, but are not limited to, the following specific numbered embodiments:

1. Culture-expanded canine progenitor cells that have a population doubling rate of less than about 24 hours, a normal karyotype, are positive for expression of telomerase and CD90, and are negative for expression of CD45 and CD34.

2. The culture-expanded canine progenitor cells of 1 above having undergone at least 40 population doublings in culture.

3. The culture-expanded canine progenitor cells of any one of 1-2 above having undergone at least 50 population doublings in culture.

4. The culture-expanded canine progenitor cells of any one of 1-3 above that have a population doubling rate of about 15 to 24 hours in culture.

5. The culture-expanded canine progenitor cells of any one of 1-4 above that have a population doubling rate of about 16 hours in culture.

6. The culture-expanded canine progenitor cells of any one of 1-5 above that are derived from bone marrow, adipose tissue, umbilical cord blood, or placental tissue.

7. The culture-expanded canine progenitor cells of any one of 1-6 above that are positive for expression of CD29.

8. The culture-expanded canine progenitor cells of any one of 1-7 above that are negative for expression of MHC Class II.

9. The culture-expanded canine progenitor cells of any one of 1-8 above that are positive for expression of one or more of PTHLH, CD13, CD44, CD49c, CD73, CD105, and IL1R2.

10. The culture-expanded canine progenitor cells of any one of 1-9 above that are positive for expression of IL1R2.

11. The culture-expanded canine progenitor cells of any one of 1-10 above that are negative for expression of rex-1 and NOV.

12. The culture-expanded canine progenitor cells of any one of 1-11 above that are positive for expression of nanog, sox-2 and oct-4.

13. The culture-expanded canine progenitor cells of any one of 1-12 above expressing telomerase up to about 55 population doublings in culture.

14. The culture-expanded canine progenitor cells of any one of 1-13 above that are capable of reducing or inhibiting T-cell proliferation in vivo and/or in vitro.

15. The culture-expanded canine progenitor cells of any one of 1-14 above that are capable of providing angiogenesis in vivo and/or in vitro.

16. The culture-expanded canine progenitor cells of any one of 1-15 above that are capable of differentiating into at least two cell types of the mesodermal germ layer.

17. The culture-expanded canine progenitor cells of any one of 1-16 above that are capable of differentiating into at least two of an osteoblast, an adipocyte, and a chondrocyte.

18. The culture-expanded canine progenitor cells of any one of 1-17 above that are not tumorigenic, do not form teratomas, are not transformed, and are not immortalized.

### BRIEF DESCRIPTION OF THE FIGURES

FIG. 1—Graph showing growth kinetics of canine multipotent adult progenitor cells (cMAPC). Every passage, cells were counted and population doublings (PD) were calculated according to the number of cells initially seeded (Cᵢ) to the number of cells harvested (Cₕ) using the following equation: PDₕ=PDᵢ+Log₂(Cₕ/Cᵢ).

FIGS. 2A-D—Flow cytometry results showing that cMAPC are positive for CD29 (FIG. 2A) and CD90 (FIG. 2B), and negative for CD45 (FIG. 2C) and MHC class II (FIG. 2D).

FIG. 3—Graph showing cMAPC expression levels of CD34, CD45, CD13, CD44, CD49c, CD73, CD90 and CD105, measured by qPCR analysis. The dotted line represents the lower limit of detection. CD34 and CD45 are not detectable. CD13, CD44, CD49c, CD73, CD90 and CD105 show positive expression.

FIGS. 4A-C—Agarose gels showing expression of pluripotency genes in cMAPC and canine mesenchymal stem cells (MSC). RNA was extracted from canine MAPC (M) and MSC(S) from 3 different donors. The RNA was converted into cDNA. The cDNA was used to perform PCR for nanog (FIG. 4A), oct4 (FIG. 4B) and sox2 (FIG. 4C). The PCR products were then loaded on a 2% agarose gel to visualize the expression of the genes. cMAPC and canine MSC show expression of nanog, oct4 and sox2.

FIG. 5—Telomerase activity in cMAPC and canine MSC. For all samples, the same number of cells was used. The assay is based on elongation of a telomere template by the endogenous telomerase in the cell sample. The amount of template is then determined by quantitative PCR. Significantly more template was elongated by telomerase in the cMAPC sample compared to the canine MSC sample from the same donor. The number between brackets represents the population doubling. Telomerase activity decreased when cells reach a higher population doubling and thus become older, as can be seen for donor 2.

FIG. 6—Results of staining to determine multi-lineage potential of cMAPC. cMAPC are able to differentiate towards osteoblasts, adipocytes and chondrocytes.

FIG. 7—Results of immunopotency assay showing that cMAPC inhibited T-cell proliferation. The immunopotency assay was performed in a 96 well round bottom plate. In each well, 100,000 canine PBMC (peripheral mononuclear blood cells) were added to cMAPC that were plated in a serial dilution ranging from 1:2 to 1:16. cPBMC were stimulated with 0.5 g/ml ConA (Concanavalin A; Sigma) and the assay was analyzed 4 days later.

FIG. 8—Results of in vitro angiogenesis assay. Conditioned medium from cMAPC induces tube formation between human umbilical vein endothelial cells (HUVEC).

FIG. 9—Cytogenic analysis showing that cMAPC have a normal karyotype.

FIGS. 10A-B—Results of microarray data for cMAPC and canine MSC. Dendrogram (FIG. 10A) and PCA (principal component analysis) plot (FIG. 10B) showed that cMAPC and cMSC form two separate clusters that can be considered as two distinct cell populations based on total gene expression.

FIGS. 11A-C—Marker expression cMAPC and canine MSC. RNA was extracted from cMAPC (M) and cMSC(S) from 3 different donors. The RNA was converted into cDNA. The cDNA was used to perform PCR for IL1R2 (FIG. 11A) and NOV (FIG. 11B). The PCR products were then loaded on a 2% agarose gel to visualize the expression of the genes. Ribosomal Protein L8 (RPL8) (FIG. 11C) was used as reference gene.

### DETAILED DESCRIPTION OF THE INVENTION

It should be understood that this invention is not limited to the particular methodology, protocols, and reagents, etc., described herein and, as such, may vary. The terminology used herein is for the purpose of describing particular embodiments only, and is not intended to limit the scope of the disclosed invention, which is defined solely by the claims.

The section headings are used herein for organizational purposes only and are not to be construed as in any way limiting the subject matter described.

The methods and techniques of the present application are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990).

### Definitions

“A” or “an” means herein one or more than one; at least one. Where the plural form is used herein, it generally includes the singular.

“Autoimmune disease” refers to the failure of a subject's immune system to distinguish self from non-self or the failure to respond to foreign antigens. The term also embraces hyperimmune responses to foreign antigens as in the case of allergic disorders. Thus, the response is present in both autoimmune disorders and allergic disorders. Autoimmune diseases include, but are not limited to, tissue injury and inflammation caused by the production of antibodies to an organism's own tissue, impaired production of cytokines and tissue damage caused by cytotoxic or non-cytotoxic mechanisms of action. In some embodiments, autoimmune diseases are inappropriately regulated immune responses that lead to patient symptoms. Typically, autoimmune responses occur when the immune system of a subject recognizes self-antigens as foreign, leading to the production of self-reactive effector immune cells. Self-reactive effector immune cells include cells from a variety of lineages, including, but not limited to, cytotoxic T-cells, helper T-cells, and B cells. While the precise mechanisms differ, the presence of autoreactive effector immune cells in a patient suffering from an autoimmune disorder may lead to the destruction of tissues and cells of the patient, resulting in pathologic symptoms. Non-limiting examples of an autoimmune disease include: immune-mediated polyarthritis; immune-mediated thrombocytopenia; keratoconjunctivitis sicca; inflammatory brain disease; and pemphigus foliaceus. Similarly, the presence of cells that undergo a hypersensitive reaction to foreign antigens to which normal individuals respond in a more restrain manner is indicative of hypersensitivity (allergy). Examples include, but are not limited to, flea allergy dermatitis, seasonal allergies, human food allergies, dog food allergies, airborne allergens, environmental allergies, home allergies, and prescription drugs. Numerous assays for determining the presence of such cells in a subject, and therefore the presence of an autoimmune disorder, such as an antigen-specific autoimmune disorder or an allergic disorder, are known to those of skill in the art and can be readily employed in the subject methods.

A “cell bank” is industry nomenclature for cells that have been grown and stored for future use. Cells may be stored in aliquots. They can be used directly out of storage or may be expanded after storage. This is a convenience so that there are “off the shelf” cells available for administration. The cells may already be stored in a pharmaceutically-acceptable excipient so they may be directly administered or they may be mixed with an appropriate excipient when they are released from storage. Cells may be frozen or otherwise stored in a form to preserve viability. In one embodiment of the invention, cell banks are created using cells produced by the methods described in this application.

“Co-administer” means to administer in conjunction with one another, together, coordinately, including simultaneous or sequential administration of two or more agents.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning.

“Comprised of” is a synonym of “comprising” (see above).

“Effective route” generally means a route which provides for delivery of an agent (e.g., canine progenitor cells) to a desired compartment, system, or location. For example, an effective route is one through which an agent can be administered to provide at the desired site of action an amount of the agent sufficient to effectuate a beneficial or desired clinical result.

“Effective time”, when referring to the invention, refers to a period of time sufficient to bring about a particular effect, such as treating an inflammatory condition or a musculoskeletal disorder.

“Immune response” refers to a patient response to foreign or self-antigens. The term includes cell-mediated, humoral, and inflammatory responses.

Use of the term “includes” is not intended to be limiting.

“Increase” or “increasing” means to induce a biological event entirely or to increase the degree of the event.

The term “inflammatory condition” refers to a disease or disorder characterized by acute or chronic inflammation. The term can refer to inflammatory diseases, such as autoinflammatory diseases (e.g., autoimmune diseases) or other inflammatory diseases. Non-limiting examples of inflammatory conditions include dermatitis (e.g., atopic dermatitis) inflammatory eye disease, inflammatory brain disease (e.g., meningoencephalomyelitis), inflammatory airway disease, and inflammatory bowel disease. Other non-limiting examples of inflammatory conditions are disclosed in U.S. Patent Application Publication No. 2006/0263337A1 to Maziarz et al., such as adverse immune reactions (e.g., those that result from other therapies), inflammatory conditions that complicate transplantation therapies (e.g., GvHD), and congenital immune disorders.

The term “ischemic condition” refers to an injury due to obstructed blood flow and reperfusion injury caused by removal of the obstruction. Non-limiting examples of ischemic conditions include acute myocardial infarction, chronic heart failure, peripheral vascular disease, stroke, chronic total occlusion, renal ischemia, and acute kidney injury.

The term “isolated” refers to cells (e.g., canine progenitor cells) that are not associated with one or more cells or one or more cellular components that are associated with the cells (e.g., canine progenitor cells) in vivo. An “enriched population” means a relative increase in numbers of a desired cell (e.g., canine progenitor cells) relative to one or more other cell types in vivo or in primary culture.

However, as used herein, the term “isolated” does not indicate the presence of only a particular cell (e.g., a canine progenitor cell). Rather, the term “isolated” indicates that the cells (e.g., canine progenitor cells) are removed from their natural tissue environment and are present at a higher concentration as compared to the normal tissue environment. Accordingly, an “isolated” cell population may further include cell types in addition to particular cells (e.g., canine progenitor cells) and may include additional tissue components. This also can be expressed in terms of cell doublings, for example. A cell (e.g., a canine progenitor cell) may be capable of undergoing at least about 10, 20, 30, 40 or more doublings in vitro or ex vivo so that it is enriched compared to its original numbers in vivo or in its original tissue environment (e.g., bone marrow, peripheral blood, placenta, umbilical cord, umbilical cord blood, adipose tissue, etc.).

“cMAPC” is an acronym for “canine multipotent adult progenitor cell” and can be used interchangeably therewith. Additionally, “cMAPC” can be used interchangeably with “canine progenitor cell”. cMAPC refers to a cell that is not an embryonic stem cell or germ cell but has some characteristics of these. cMAPC can be characterized in a number of different features including, but not limited to, having a population doubling rate of less than about 24 hours in culture, having extended replicative capacity in culture and a normal karyotype, giving rise to cell progeny of more than two cell types of the mesodermal germ layer upon differentiation (e.g., osteoblast, adipocyte or chondrocyte), and/or although they are post-natal somatic cells, they may express markers of these primitive cell types, such as nanog, sox-2 and oct-4. Culture-expanded cMAPCs may also express one or more of parathyroid hormone-like hormone (PTHLH), CD13, CD44, CD49c, CD73, CD90, CD105, and interleukin 1 receptor type 2 (IL1R2), and be negative for expression of rex-1, CD34, CD45, and nephroblastoma overexpressed (NOV). Further, culture-expanded cMAPCs may be surface antigen positive for CD90 and CD29 and surface antigen negative for CD45 and MHC Class II. Fifth, like a stem cell, cMAPCs may self-renew; that is, have an extended replication capacity in culture without being transformed. This means that these cells express telomerase (i.e., have telomerase activity) in culture. Sixth, culture-expanded MAPCs are not tumorigenic, do not form teratomas, are not transformed, and are not immortalized. Accordingly, the cell type that was designated “cMAPC” may be characterized by alternative basic characteristics that describe the cell via some of its novel properties.

The term “adult” in cMAPC is non-restrictive. It refers to a non-embryonic cell, such as a post-natal somatic cell.

The term “musculoskeletal disorder” includes all disorders related to bone, muscle, ligaments, tendons, cartilage and joints. Treatment of a musculoskeletal disease or disorder is within the ambit of regenerative medicine. For example, disorders requiring spinal fixation, spinal stabilization, repair of segmental defects in the body (such as in long bones and flat bones), disorders of the vertebrae and discs including, but not limited to, disruption of the disc annulus such as annular fissures, chronic inflammation of the disc, localized disc herniations with contained or escaped extrusions, and relative instability of the vertebrae surrounding the disc are musculoskeletal disorders. Musculoskeletal disorders also include sprains, strains and tears of ligaments (e.g., complete or partial cruciate ligament rupture), tendons, muscles (e.g., skeletal muscles and myocardium) and cartilage, tendonitis, tenosynovitis, fibromyalgia, osteoarthritis, rheumatoid arthritis, polymyalgia rheumatica, bursitis, and osteoporosis. In addition, musculoskeletal disorders include genetic diseases of the musculoskeletal system as well as musculoskeletal aspects of lysosomal storage diseases.

“Negative expression”, when referring to a protein or nucleic acid (e.g., mRNA), means that the protein or nucleic acid is absent in a sample and/or not present in a sample at a level that is detectable by a known assay as compared to a control sample.

“Pharmaceutically acceptable carrier” is any pharmaceutically acceptable medium for the canine progenitor cells used in the present disclosure. Such a medium may retain isotonicity, cell metabolism, pH, and the like. It is compatible with administration to a subject in vivo, and can be used, therefore, for cell delivery and treatment.

“Population doubling rate” refers to the amount of cell population doubling per unit of time. Cell population doublings (PD) can be calculated according to the following equation: PDₕ=PDᵢ+Log₂(Cₕ/Cᵢ); where Cᵢ represents the cells initially seeded and Cₕ represents the number of cells harvested. In the case of the culture-expanded canine progenitor cells, the population doubling rate is less than about 24 hours, for example, about 15 to 24 hours, about 15 hours, about 16 hours (e.g., 16 hours), about 17 hours, about 18 hours, about 19 hours, about 20 hours, about 21 hours, about 22 hours, about 23 hours, or about 24 hours in culture throughout the expansion process, such as from about 10 population doublings to about 55 population doublings.

“Positive expression”, when referring to a protein or nucleic acid (e.g., mRNA), means that the protein or nucleic acid is present in a sample at a level that is detectable by a known assay as compared to a control sample.

The term “reduce” as used herein means to prevent as well as decrease. In the context of treatment, to “reduce” is to either prevent or ameliorate one or more clinical symptoms. A clinical symptom is one (or more) that has or will have, if left untreated, a negative impact on the quality of life (health) of the subject. In an in vitro context, to “reduce” is to decrease one or more analytes or biomarkers, which may be assayed and then correlated to a particular outcome or endpoint.

“Self-renewal” of a stem cell refers to the ability to produce replicate daughter stem cells having differentiation potential that is identical to those from which they arose.

“Subject” means an animal that is a member of the family Canidae, which includes wolves, jackals, foxes, coyote, and the domestic dog (Canis lupus familiaris). Thus, any one of the terms “dog”, “canine”, or “canid” can be used interchangeably when referring to a subject of the present application. A canid may be a domestic dog, a wolf, or an animal that has some genetic contribution(s) from more than one species of the family Canidae. Thus, a canine of the present application can include any purebred dog or mixed breed.

“Substantially pure” refers to a population of canine progenitor cells (i.e., cMAPCs) that is free or substantially free of other cell types. Cell purification can be accomplished by any means known to one of ordinary skill in the art. For example, a substantially pure population of canine progenitor cells (i.e., cMAPCs) can be achieved by growth of canine progenitor cells (i.e., cMAPCs) or by selection from a less pure population. A culture of canine progenitor cells (i.e., cMAPCs) is substantially pure if at least 85%, 90%, 95%, 96%, 97%, 98%, 99%, or 100% of the growing cells in the culture are canine progenitor cells (i.e., cMAPCs). The presence of only a small percentage or zero percentage of other growing cell types in a culture of canine progenitor cells (i.e., cMAPCs) means the culture is a substantially pure culture of canine progenitor cells (i.e., cMAPCs).

“Suppression,” “inhibition” and “prevention,” when used in the context of an immune response, are used herein in accordance with accepted definitions. For example, “suppression” results when an ongoing immune response (e.g., aberrant T-cell activity, such as proliferation) is blocked or significantly reduced as compared with the level of immune response that results in the absence of treatment, e.g., by the cells disclosed herein. “Inhibition” refers to blocking the occurrence of an immune response or significantly reducing such response as compared with the level of immune response that results absent treatment, e.g., by the cells disclosed herein. When administered prophylactically, such blockage may be complete so that no targeted immune response occurs, typically referred to as a “prevention” with regard to completely blocking the immune response before onset; or in the present disclosure, the treatment may reduce the effect as compared to the normal untreated state, typically referred to as suppression or inhibition.

The term “therapeutically effective amount” refers to the amount of an agent (e.g., culture-expanded canine progenitor cells) determined to produce any therapeutic response in a subject. For example, effective anti-inflammatory therapeutic agents may prolong the survivability of the subject, and/or inhibit overt clinical symptoms. Treatments that are therapeutically effective within the meaning of the term as used herein include treatments that improve a subject's quality of life even if they do not improve the disease outcome per se. Such therapeutically effective amounts are readily ascertained by one of ordinary skill in the art. Thus, to “treat” means to deliver such an amount. In some instances, treating can prevent or ameliorate any pathological symptom(s) of an inflammatory condition (e.g., an autoimmune disease) or a musculoskeletal disorder.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

### Selection and Phenotype of Culture-Expanded Canine Progenitor Cells

The present invention provides culture-expanded canine progenitor cells (i.e., cMAPC), isolated from adult canines, that can differentiate to form at least two cell types of the mesodermal germ layer, such as osteoblasts, adipocytes, and chondrocytes. These cells are also capable of extended replication in culture and exhibit one or more of the following through about 55 population doublings: a population doubling rate of less than about 24 hours, a normal karyotype; and express markers of extended replication (e.g., telomerase) and pluripotency (e.g., oct4).

The canine progenitor cells described herein were isolated and expanded by the inventors, who identified a number of specific cell surface and other phenotypic markers that characterize the cells. The methods described below can be used to isolate and grow canine progenitor cells from any adult canine tissue, such as bone marrow, adipose tissue, umbilical cord blood, or placental tissue. As such, it is possible for one of ordinary skill in the art to obtain tissue from a canine and select culture-expanded canine progenitor cells using known positive or negative selection techniques, relying upon certain surface and/or genetic markers expressed (or not expressed) on these cells, as identified by the inventors, without undue experimentation.

### 1. Phenotype of Culture-Expanded Canine Progenitor Cells

In one embodiment, culture-expanded canine progenitor cells (i.e., cMAPCs) are provided.

The inventors have discovered that the canine progenitor cells of the present application (i.e., cMAPCs) have a doubling rate of less than about 24 hours in culture, which is surprisingly less than other canine progenitor cells, such as canine mesenchymal stem cells (cMSCs). As such, in some instances, the canine progenitor cells (i.e., cMAPCs) have a population doubling rate of about 15 hours, about 16 hours, about 17 hours, about 18 hours, about 19 hours, about 20 hours, about 21 hours, about 22 hours, about 23 hours, or about 24 hours, e.g., less than 24 hours in culture. In some instances, the population doubling rate is less than about 24 hours through about 10-15 population doublings in culture, through about 15-20 population doublings in culture, through about 20-25 population doublings in culture, through about 25-30 population doublings in culture, through about 30-35 population doublings in culture, through about 35-40 population doublings in culture, through about 40-45 population doublings in culture, through about 45-50 population doublings in culture, or through about 50-55 population doublings in culture.

The culture-expanded canine progenitor cells (i.e., cMAPCs) of the present application have a normal karyotype. A “karyotype” refers to the number and appearance of chromosomes in the nucleus of a eukaryotic cell. The term is also used to designate a complete set of chromosomes in a species or organism. Karyotypes describe the number of chromosomes and their appearance via light microscopy. A karyotype is established with respect to length, position of centromeres, banding pattern, and other physical characteristics. Accordingly, a karyotype is considered normal when there are no apparent aneuploidies in the chromosomes.

Chromosomal aberrations are established by standard procedures in the art, namely, staining with a suitable dye, such as Giemsa (“G-banding”). Such banding is obtained following limited digestion of chromosomes with trypsin. This yields a series of lightly and darkly stained bands where the dark regions tend to be heterochromatic and the light regions euchromatic. Each chromosome has a characteristic banding pattern that helps to identify it. And both chromosomes in a diploid nucleus will have the same banding pattern.

Chromosome abnormalities are routinely detectable. They can be numerical, such as extra or missing chromosomes, or structural, such as translocations, inversions, large scale deletions, and duplications. These can be detected by various routine banding techniques, such as G-banding. Occasionally, technical artifacts that are associated with the processing of chromosomes can generate apparent differences between two homologs (of the same chromosome). But these artifacts are routinely identified by analyzing an accepted number of metaphase spreads from the individual, for example, around 15-20. Given that level of analysis, it is highly unlikely that the same technical artifact would repeatedly occur in a given specimen. For a discussion of karyotyping for chromosomal abnormalities, see O'Connor, C. (2008) Karyotyping for chromosomal abnormalities. Nature Education 1(1):27.

The canine progenitor cells (i.e., cMAPCs) of the present application have a normal karyotype through about 10-15 population doublings in culture, through about 15-20 population doublings in culture, through about 20-25 population doublings in culture, through about 25-30 population doublings in culture, through about 30-35 population doublings in culture, through about 35-40 population doublings in culture, through about 40-45 population doublings in culture, through about 45-50 population doublings in culture, or through about 50-55 population doublings in culture.

The canine progenitor cells (i.e., cMAPCs) can differentiate into at least two cell types of the mesodermal germ layer through about 10-15 population doublings in culture, through about 15-20 population doublings in culture, through about 20-25 population doublings in culture, through about 25-30 population doublings in culture, through about 30-35 population doublings in culture, through about 35-40 population doublings in culture, through about 40-45 population doublings in culture, through about 45-50 population doublings in culture, or through about 50-55 population doublings in culture. Cell types of mesodermal germ layer into which the culture-expanded canine progenitor cells (i.e., cMAPCs) of the present invention can differentiate include adipocytes, osteoblasts and chondrocytes. In another example, the canine progenitor cells (i.e., cMAPCs) can differentiate into three or more cell types of the mesodermal germ layer through about 10-15 population doublings in culture, through about 15-20 population doublings in culture, through about 20-25 population doublings in culture, through about 25-30 population doublings in culture, through about 30-35 population doublings in culture, through about 35-40 population doublings in culture, through about 40-45 population doublings in culture, through about 45-50 population doublings in culture, or through about 50-55 population doublings in culture.

The canine progenitor cells (i.e., cMAPCs) are characterized by extended replication in culture. As such, the canine progenitor cells (i.e., cMAPCs) have undergone, or are capable of undergoing, at least 10, at least 20, at least 30, at least 40, or at least 50 or more population doublings in culture. In one example, the canine progenitor cells (i.e., cMAPCs) have undergone, or are capable of undergoing, 50 population doublings in culture.

The culture-expanded canine progenitor cells (i.e., cMAPCs) are characterized by positive or negative expression of certain molecular markers, such as cell surface, genetic, and functional markers. Non-limiting examples of these markers are disclosed below. In some instances, culture-expanded canine progenitor cells (i.e., cMAPCs) are characterized by positive or negative expression of certain molecular markers through about 50 population doublings, for example, through about 30-50 population doublings, about 30-35 population doublings, about 35-40 population doublings, about 40-45 population doublings, or about 45-50 population doublings. In one example, culture-expanded canine progenitor cells (i.e., cMAPCs) are characterized by positive or negative expression of certain molecular markers through about 40 or 44 population doublings.

In one example, the culture-expanded canine progenitor cells (i.e., cMAPCs) are surface antigen positive for at least one of CD90 and CD29, and/or surface antigen negative for at least one of CD34, CD45 and MHC Class II.

In one example, the culture-expanded canine progenitor cells (i.e., cMAPCs) are surface antigen positive for CD90 and surface antigen negative for CD34 and CD45.

In another example, the culture-expanded canine progenitor cells (i.e., cMAPCs) are positive for expression of one or more of PTHLH, CD13, CD44, CD49c, CD73, CD90, CD105, IL1R2, nanog, oct4 and sox-2, and/or negative for expression of rex-1, CD34, CD45, and NOV.

In another example, the culture-expanded canine progenitor cells (i.e., cMAPCs) are positive for expression of IL1R2.

In another example, the culture-expanded canine progenitor cells (i.e., cMAPCs) are positive for telomerase activity. Culture-expanded canine progenitor cells (i.e., cMAPCs) are positive for telomerase activity through about 50 population doublings, for example, through about 20-50 population doublings, about 20-25 population doublings, about 25-30 population doublings, about 30-35 population doublings, about 35-40 population doublings, about 40-45 population doublings, or about 45-50 population doublings. In one example, culture-expanded canine progenitor cells (i.e., cMAPCs) are positive for telomerase activity through about 40 or 44 population doublings.

In another example, the culture-expanded canine progenitor cells (i.e., cMAPCs) are positive for oct-4 expression. Culture-expanded canine progenitor cells (i.e., cMAPCs) are positive for oct-4 expression through about 50 population doublings, for example, through about 20-50 population doublings, about 20-25 population doublings, about 25-30 population doublings, about 30-35 population doublings, about 35-40 population doublings, about 40-45 population doublings, or about 45-50 population doublings. In one example, culture-expanded canine progenitor cells (i.e., cMAPCs) are positive for oct-4 expression through about 40 or 44 population doublings.

In another example, the culture-expanded canine progenitor cells (i.e., cMAPCs) can differentiate into at least two cell types of the mesodermal germ layer. Culture-expanded canine progenitor cells (i.e., cMAPCs) differentiate into at least two cell types of the mesodermal germ layer through about 50 population doublings, for example, through about 20-50 population doublings, about 20-25 population doublings, about 25-30 population doublings, about 30-35 population doublings, about 35-40 population doublings, about 40-45 population doublings, or about 45-50 population doublings. In one example, culture-expanded canine progenitor cells (i.e., cMAPCs) differentiate into at least two cell types of the mesodermal germ layer through about 40 or 44 population doublings.

In another example, the culture-expanded canine progenitor cells (i.e., cMAPCs) have the ability to reduce or inhibit T-cell proliferation in vivo and/or in vitro.

In another example, the canine progenitor cells (i.e., cMAPCs) have the ability to induce or promote angiogenesis in vivo and/or in vitro.

As such, the skilled artisan will appreciate that the nature of the culture-expanded canine progenitor cells (i.e., cMAPCs) can be ascertained, as well as the purity of the cells based on the presence or absence of one or a combination of the molecular and/or functional markers discussed above.

### 2. Isolation and Expansion of Canine Progenitor Cells

In one embodiment, canine progenitor cells (i.e., cMAPCs) of the present invention can be isolated from multiple tissue sources, including, but not limited to, bone marrow, placenta, umbilical cord and cord blood, muscle, brain, liver, spinal cord, blood or skin. In one example, bone marrow aspirate is obtained from the femur or tibia of an adult canine subject using a syringe (e.g., a Jamshidi needle). In the exemplary material, the canine progenitor cells (i.e., cMAPCs) are derived from bone marrow.

In some instances, cells obtained from the tissue source can be fractionated using, for example, Histopaque density centrifugation. The mononuclear fraction can be collected and a total cell count determined. Cells of the mononuclear fraction can then be inoculated on or into a static (e.g., a protein-coated flask) or non-static culture vessel (e.g., a stirred-tank bioreactor) at a desired density and cultured in a culture medium under conditions sufficient to expand the cells. In one embodiment, cells of the mononuclear fraction are plated onto a flask coated with CPPT. Cell of the mononuclear fraction can be plated (e.g., on a protein-coated flask) at a density of about 500 cells/cm² to about 300,000 cells/cm² or greater, for example, about 100,000 cells/cm² to about 250,000 cells/cm², about 40,000 cells/cm² to about 100,000 cells/cm², or about 2,000 cells/cm² to about 5,000 cells/cm². In another embodiment, cells of the mononuclear fraction can be inoculated into a non-static culture vessel, such as a hollow fiber bioreactor at a desired density and cultured in a culture medium under conditions sufficient to expand the cells. One example of a hollow fiber bioreactor is described in U.S. Patent Application Publication No. U.S. 2012/0308531A1 to Pinxteren et al., and is also commercially available as the Quantum® cell expansion system (Terumo, BCT, Lakewood, CO).

Cells of the mononuclear fraction can be cultured in a culture medium containing serum as well as other supplements necessary for cell growth and survival (e.g., growth factors, amino acids, sugars, hormones, buffering agents, vitamins, etc.). In one example, cells of the mononuclear fraction can be cultured in a static culture vessel (e.g., a protein-coated flask) using a culture medium comprising the following components: about 20-60% MCDB-201 medium (e.g., about 40%); about 20-60% αMEM medium (e.g., about 35-50%); about 1-5 mM Ultraglutamine (e.g., about 2 mM); about 5-20% FBS (e.g., about 10-18%); about 0.5-2×ITS (insulin-transferrin-selenium) (e.g., about 1×); about 0.1-2×LA-BSA (linoleic acid-bovine serum albumin (e.g., about 0.5×); about 5-150 UM L-Ascorbic acid-2-phosphate (e.g., about 100 μM); about 5-20 ng/ml human/canine PDGF-BB (e.g., about 10 ng/ml); about 10-75 mM dexamethasone (e.g., about 50 nM); about 5-20 ng/ml canine EGF (e.g., about 10 ng/ml); and about 0.5-20 ng/ml hFGF2 (e.g., about 1-10 ng/ml).

In another embodiment, cells of the mononuclear fraction can be cultured in a non-static culture vessel (e.g., a hollow fiber bioreactor) using a culture medium comprising the following components: about 20-60% MCDB-201 medium (e.g., about 40%); about 20-60% αMEM medium (e.g., about 35-50%); about 1-5 mM Ultraglutamine (e.g., about 2 mM); about 5-20% FBS (e.g., about 10-18%); about 0.5-2×ITS (insulin-transferrin-selenium) (e.g., about 1×); about 0.1-2×LA-BSA (linoleic acid-bovine serum albumin (e.g., about 0.5×); about 5-150 μM L-Ascorbic acid-2-phosphate (e.g., about 100 μM); about 5-20 ng/ml human/canine PDGF-BB (e.g., about 10 ng/ml); about 10-75 mM dexamethasone (e.g., about 50 nM); about 5-20 ng/ml canine EGF (e.g., about 10 ng/ml); about 0.5-20 ng/ml hFGF2 (e.g., about 1-10 ng/ml); and about 0.5-20 ng/ml TGFβ1 (e.g., about 1-10 ng/ml).

Cells of the mononuclear fraction can be incubated in a humidified incubator under conditions sufficient to expand the cells to a desired confluency. In some instances, cells are incubated at about 38° C. for a period of 1-2 days, 2-3 days, 3-4 days, 4-5 days, or 5 or more days. Cells are also incubated at a desired CO₂ concentration (e.g., about 1-2%, about 2-3%, about 3-4%, about 4-5%, or about 5% or more) and a desired O₂ concentration (for example, about 1-10%, e.g., about 3-5%). Cells can be lifted and passaged when a desired confluency is reached. In some instances, cells can be lifted and passaged at a confluency of less than 100%, for example, a confluency of about 10-20%, about 20-30%, about 30-40%, about 40-50%, about 50-60%, about 60-70%, about 70-80%, about 80-90%, or about 90-99%. In one example, cells are lifted and passaged at a confluency of 50-80%.

In one embodiment, canine progenitor cells are isolated and expanded as follows. Bone marrow aspirate can be obtained from the femur or tibia of a canine. The bone marrow aspirate can be drawn into a syringe using a Jamshidi needle. The mononuclear fraction can be isolated by Histopaque density centrifugation. Total cell count can be determined and the cells then plated at a density of about 100,000 to 250,000 cells/cm² in a CPPT-coated flask containing a culture medium. The culture medium can include the following components: about 40% MCDB-201 medium; about 35-50% αMEM medium; about 2 mM Ultraglutamin; about 10-18% FBS; about 1×ITS (insulin-transferrin-selenium); about 0.5×LA-BSA (linoleic acid-bovine serum albumin); about 100 μM L-Ascorbic acid-2-phosphate; about 10 ng/ml human/canine PDGF-BB; about 50 nM dexamethasone; about 10 ng/ml canine EGF; about 1-10 ng/ml hFGF2; and about 1-10 ng/ml TGFβ1. Flasks can be incubated in a humidified incubator at 38° C., 5% CO₂, 5% O₂. After about 3 to 5 days, clonal expansion of the cells is visible. When clones reach confluency of about 50-80%, the cells can be lifted and passaged.

Next, cells can be washed with PBS and afterwards detached from the plates using trypsin. The trypsinization reaction can be stopped by adding Dulbecco's Phosphate Buffered Saline (DPBS) to the flasks. The cell solution can then be transferred to a conical tube and centrifuged at about 500 ×g for about 5 minutes. Next, the supernatant can be removed and the cell pellet resuspended in DPBS. Cell number can be determined and the cells then seeded at a density of about 2,000 cells/cm² in CPPT-coated flasks in a culture medium as described above. Cells can be incubated at 38° C., 5% CO₂, 5% O₂ and passaged every 2 to 3 days.

In one embodiment, the expanded cells are subject to one or a combination of known positive or negative selection techniques that rely upon the molecular markers and/or potencies (discussed above) that are expressed (or not expressed) or exhibited in these cells. As such, culture-expanded canine progenitor cells (i.e., cMAPCs) of the present invention can be selected based on the presence and/or absence of one or more markers (as disclosed herein) and/or potencies using the selection techniques discussed below.

Both positive and negative selection techniques are available to those of skill in the art, and numerous monoclonal and polyclonal antibodies suitable for negative selection purposes are also available in the art (see, for example, Leukocyte Typing V, Schlossman, et al., Eds. (1995) Oxford University Press) and are commercially available from a number of sources.

Techniques for mammalian cell separation from a mixture of cell populations have also been described by Schwartz, et al., in U.S. Pat. No. 5,759,793 (magnetic separation), Basch et al., 1983 (immunoaffinity chromatography), and Wysocki and Sato, 1978 (fluorescence-activated cell sorting, FACS).

In one embodiment, culture-expanded canine progenitor cells are selected that express (are positive for) at least one of CD90 and/or do not express (are negative for) at least one of CD45 and CD34. In some instances, FACS is used to detect the presence or absence of cell surface antigens. As described in the Example below, for instance, FACS can be used to detect the presence or absence of CD90, CD45, and CD34 and as well MHC Class II and CD29.

In another embodiment, culture-expanded canine progenitor cells are selected that express (are positive for) one or more of PTHLH, CD13, CD44, CD49c, CD73, CD90, CD105, IL1R2, nanog, oct4, and sox-2, and/or do not express (are negative for) expression of rex-1, CD34, CD45, and NOV. In some instances, PCR (e.g., semi-quantitative PCR, sqPCR) is used to detect the presence or absence of genetic markers. As described in the Example below, for instance, sqPCR can be used to detect the presence or absence of PTHLH, CD13, CD44, CD49c, CD73, CD90, CD105, IL1R2, nanog, oct4, sox-2, rex-1, CD34, CD45 and NOV.

In another embodiment, culture-expanded canine progenitor cells are selected that are positive for telomerase activity. Assays for detection of telomerase activity are known in the art. For any assay of telomerase activity, it is important that a positive and negative control must be included. In one example, as described in the Example below, telomerase activity can be determined using a commercially available kit, such as the TRAPeze RT Telomerase detection kit (Merck).

In another embodiment, culture-expanded canine progenitor cells are selected that have the ability to reduce or inhibit T-cell proliferation in vivo and/or in vitro. The ability of the cells to inhibit or reduce T-cell proliferation can be determined using an immunopotency assay, examples of which are known in the art. One example of an immunopotency assay used to determine the ability of cells to reduce or inhibit T-cell proliferation is provided in the Example below. Other examples of immunopotency assays, such as a mixed lymphocyte reaction (MLR), are disclosed in U.S. Patent Application Publication No. 2006/0263337A1 to Maziarz et al.

In another embodiment, culture-expanded canine progenitor cells are selected that have the ability to induce or promote angiogenesis in vivo and/or in vitro. Assays for determining the ability of cells to induce or promote angiogenesis are known in the art. In one example, as described in U.S. Patent Application Publication No. US 2014/0242629A1 to Woda et al. and the Example below, a HUVEC tube formation assay can be used to determine the ability of the cells to induce or promote angiogenesis.

In some embodiments, the purity of the selected, culture-expanded canine progenitor cells (i.e., cMAPCs) is about 100% (substantially pure). In other embodiments, it is 95% to 100%. In some embodiments, it is 85% to 95%. In further embodiments, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. In another embodiment, purity can be expressed in terms of cell doublings where the canine cells (i.e., cMAPCs) have undergone, for example, 1-5, 5-10, 10-20, 20-30, 30-40, 40-50 or more cell doublings in culture. In one example, the canine progenitor cells (i.e., cMAPCs) have undergone at least 40, and preferably at least 50, population doublings in culture.

Selected canine progenitor cells (i.e., cMAPCs) may be further cultured in static or non-static culture vessels, as described above.

### Compositions

In one embodiment, a composition can comprise culture-expanded canine progenitor cells (i.e., cMAPCs) of the present invention and a second component (e.g., an additive, vehicle or carrier, such as a culture medium or media).

In another embodiment, culture-expanded canine progenitor cells (i.e., cMAPCs) of the present invention can be formulated as a pharmaceutical composition.

U.S. Pat. No. 7,015,037 is incorporated by reference for teaching pharmaceutical formulations. In certain embodiments, the culture-expanded canine progenitor cells (i.e., cMAPCs) of the present invention are present within a composition adapted for and suitable for delivery, i.e., physiologically compatible.

In some embodiments, the purity of the culture-expanded canine progenitor cells (i.e., cMAPCs) for administration to a subject is about 100% (substantially pure). In other embodiments it is 95% to 100%. In some embodiments it is 85% to 95%. Particularly, in the case of admixtures with other cells, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. Or isolation/purity can be expressed in terms of cell doublings where the canine cells have undergone, for example, 1-5, 5-10, 10-20, or more cell doublings in culture.

The choice of formulation for administering the culture-expanded canine progenitor cells (i.e., cMAPCs) of the present invention for a given application will depend on a variety of factors. Prominent among these will be the species of subject, the nature of the inflammatory condition or musculoskeletal disorder being treated, its state and distribution in the subject, the nature of other therapies and agents that are being administered, the optimum route for administration, survivability via the route, the dosing regimen, and other factors that will be apparent to those skilled in the art. For instance, the choice of suitable carriers and other additives will depend on the exact route of administration and the nature of the particular dosage form.

Final formulations of the aqueous suspension of culture-expanded canine progenitor cells (i.e., cMAPCs)/medium will typically involve adjusting the ionic strength of the suspension to isotonicity (i.e., about 0.1 to 0.2) and to physiological pH (i.e., about pH 6.8 to 7.5). The final formulation will also typically contain a fluid lubricant.

In some embodiments, the culture-expanded canine progenitor cells (i.e., cMAPCs) of the present invention are formulated in a unit dosage injectable form, such as a solution, suspension, or emulsion. Pharmaceutical formulations suitable for injection of the cells typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of culture-expanded canine progenitor cells (i.e., cMAPCs) and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

In some embodiments, culture-expanded canine progenitor cells (i.e., cMAPCs) of the present invention are encapsulated for administration, particularly where encapsulation enhances the effectiveness of the therapy, or provides advantages in handling and/or shelf life. Culture-expanded canine progenitor cells (i.e., cMAPCs) may be encapsulated by membranes, as well as capsules, prior to implantation. It is contemplated that any of the many methods of cell encapsulation available may be employed.

A wide variety of materials may be used in various embodiments for microencapsulation of canine cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers.

Techniques for microencapsulation of cells that may be used for administration of cells are known to those of skill in the art and are described, for example, in Chang, P., et al., 1999; Matthew, H. W., et al., 1991; Yanagi, K., et al., 1989; Cai Z. H., et al., 1988; Chang, T. M., 1992 and in U.S. Pat. No. 5,639,275 (which, for example, describes a biocompatible capsule for long-term maintenance of cells that stably express biologically active molecules). Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of cells.

Certain embodiments incorporate culture-expanded canine cells (i.e., cMAPCs) into a polymer, such as a biopolymer or synthetic polymer. Examples of biopolymers include, but are not limited to, fibronectin, fibrin, fibrinogen, thrombin, collagen, and proteoglycans. Other factors, such as the cytokines discussed above, can also be incorporated into the polymer. In other embodiments of the invention, culture-expanded canine progenitor cells (i.e., cMAPCs) may be incorporated in the interstices of a three-dimensional gel. A large polymer or gel, typically, will be surgically implanted. A polymer or gel that can be formulated in small enough particles or fibers can be administered by other common, more convenient, non-surgical routes.

The dosage of the culture-expanded canine progenitor cells (i.e., cMAPCs) will vary within wide limits and will be fitted to the individual requirements in each particular case. The number of cells will vary depending on the weight and condition of the recipient, the number or frequency of administrations, and other variables known to those of skill in the art. The culture-expanded canine cells (i.e., cMAPCs) can be administered by a route that is suitable for the tissue or organ. For example, culture-expanded canine progenitor cells (i.e., cMAPCs) can be administered systemically, i.e., by intravenous administration, or can be targeted to a particular tissue or organ, such as the brain or spinal cord, by intrathecal administration.

The culture-expanded canine progenitor cells (i.e., cMAPCs) of the present invention can be suspended in an appropriate excipient in a concentration from about 0.01 to about 5×10⁶ cells/ml or more. Suitable excipients for injection solutions are those that are biologically and physiologically compatible with the cells and with the recipient, such as buffered saline solution or other suitable excipients. The composition for administration can be formulated, produced, and stored according to standard methods complying with proper sterility and stability.

### Dosing

Doses for canine subjects can be determined without undue experimentation by the skilled artisan, from this disclosure, the documents cited herein, and the knowledge in the art. The dose of culture-expanded canine progenitor cells (i.e., cMAPCs) appropriate to be used in accordance with various embodiments of the invention will depend on numerous factors. The parameters that will determine optimal doses to be administered for primary and adjunctive therapy generally will include some or all of the following: the inflammatory or musculoskeletal condition being treated and its stage; the health, gender, age, weight, and metabolic rate of the subject; the subject's immunocompetence; other therapies being administered; and expected potential complications from the subject's history or genotype. The parameters may also include: whether the culture-expanded canine progenitor cells (i.e., cMAPCs) are syngeneic, autologous, allogeneic, or xenogeneic; the site and/or distribution that must be targeted for the cells/medium to be effective; and such characteristics of the site such as accessibility to cells/medium and/or engraftment of cells. Additional parameters include co-administration with other factors (such as growth factors and cytokines). The optimal dose in a given situation also will take into consideration the way in which the culture-expanded canine progenitor cells (i.e., cMAPCs) are formulated, the way they are administered, and the degree to which the culture-expanded canine progenitor cells will be localized at the target sites following administration.

In various embodiments, culture-expanded canine progenitor cells (i.e., cMAPCs) of the present invention may be administered in an initial dose, and thereafter maintained by further administration. Culture-expanded canine progenitor cells (i.e., cMAPCs) may be administered by one method initially, and thereafter administered by the same method or one or more different methods. The levels can be maintained by the ongoing administration of the culture-expanded canine cells (i.e., cMAPCs). Various embodiments administer the culture-expanded canine progenitor cells (i.e., cMAPCs) either initially or to maintain their level in the subject or both by intravenous injection. In a variety of embodiments, other forms of administration are used, dependent upon the subject's condition and other factors, discussed elsewhere herein.

Culture-expanded canine progenitor cells (i.e., cMAPCs) may be administered in many frequencies over a wide range of times. Generally lengths of treatment will be proportional to the length of the disease process, the effectiveness of the therapies being applied, and the condition and response of the subject being treated.

### Uses

Administering the culture-expanded canine progenitor cells (i.e., cMAPCs) of the present invention is useful to reduce any of the overt symptoms of an inflammatory condition or musculoskeletal disorder as described in this application. This may be based on underlying effects of the cells, such as: treating inflammatory conditions (e.g., autoimmune diseases) by reducing or inhibiting aberrant T-cell proliferation; treating ischemic conditions (e.g., myocardial infarction) by promoting angiogenesis; and treating musculoskeletal disorders (e.g., cruciate ligament rupture) by differentiating into connective tissues, such as ligament, tendon, bone, muscle and cartilage.

In one example, the compositions and methods disclosed herein are directed to treating an inflammatory condition in a subject by administering the culture-expanded canine progenitor cells (i.e., cMAPCs) compositions disclosed herein. In some embodiments, the subject is suffering from an autoimmune disease, and the cell compositions are used to treat the autoimmune disease.

In another example, the compositions and methods disclosed herein are directed to treating a musculoskeletal disorder in a subject by administering the culture-expanded canine progenitor cells (i.e., cMAPCs) compositions disclosed herein. In one embodiment, the subject is suffering from osteoarthritis, and the culture-expanded canine progenitor cell (i.e., cMAPC) compositions are used to treat the osteoarthritis. In another embodiment, the subject is suffering from a cruciate ligament rupture, and the culture-expanded canine progenitor cell (i.e., cMAPC) compositions are used to treat the cruciate ligament rupture. In yet another embodiment, the subject is suffering from a spinal condition, and the culture-expanded canine progenitor cell (i.e., cMAPC) compositions are used to treat the spinal condition.

In another example, the compositions and methods disclosed herein are directed to treating an ischemic condition in a subject by administering the culture-expanded canine progenitor cell (i.e., cMAPC) compositions disclosed herein. In one embodiment, the subject is suffering from acute myocardial infarction, chronic heart failure, peripheral vascular disease, stroke, chronic total occlusion, renal ischemia and/or acute kidney injury, and the culture-expanded canine progenitor cell (i.e., cMAPC) compositions are used to treat the acute myocardial infarction, chronic heart failure, peripheral vascular disease, stroke, chronic total occlusion, renal ischemia and/or acute kidney injury.

The compositions disclosed herein are used to treat, alleviate or ameliorate the symptoms, or suppress a wide variety of inflammatory conditions, ischemic conditions, and musculoskeletal disorders, such as those described above.

In some embodiments, culture-expanded canine progenitor cells (i.e., cMAPCs) of the present invention may be used to deliver suppressive or other biologic factors to sites disease or damage (e.g., sites of inflammation or ischemia), such as, but not limited to cytokines, stem cell growth factors, and angiogenesis regulators. For example, in some embodiments, culture-expanded canine progenitor cells (i.e., cMAPCs) can be transduced with genes encoding a desired biological factor, which the cells will then produce once within the subject, e.g., at the site of inflammation or ischemia.

In some embodiments, the culture-expanded canine progenitor cells (i.e., cMAPCs) compositions disclosed herein can be used to treat infectious diseases in which, e.g., the pathogenicity of the infection is not a result of the cytopathic effects of the pathogen, but rather the tissue damage caused by the immunoinflammatory response to the infectious agent. In diseases, such as hepatitis B or C or HSV-induced corneal inflammation, therapy with the culture-expanded canine progenitor cells (i.e., cMAPCs) disclosed herein provides a unique opportunity to control viral-induced immunoinflammatory disease. Viruses, such as Coxsackie, are known to cause pancreatitis and have been associated with the development of Type 1 Diabetes. Thus, culture-expanded canine progenitor cell (i.e., cMAPCs) compositions as disclosed herein can be used to suppress local tissue damage caused by the infection and reduce the inflammation that incites autoimmune disorder development.

The subject methods find use in the treatment of a variety of different conditions and transplant situations. To keep the culture-expanded canine progenitor cells (i.e., cMAPCs) at the site until completion of the surgical procedure, in some embodiments, it is convenient to administer the culture-expanded canine progenitor cells (i.e., cMAPCs) in a pharmaceutically acceptable carrier, such as an artificial gel, or in clotted plasma, or by utilizing other controlled release mechanism known in the art.

In addition, other uses provided by the present invention include screening one or more agents or compounds for the ability to affect the ability or potency of culture-expanded canine progenitor cells (i.e., cMAPCs) to have one or more of the effects discussed above, such as providing angiogenesis and reducing or inhibiting T-cell proliferation in vitro and/or in vivo. Such a screening method includes (i) contacting culture-expanded canine progenitor cells (i.e., cMAPCs) with an agent or compound, and (ii) assessing the ability or potency of the cells to have an effect. Such agents include, but are not limited to, small organic molecules, antisense nucleic acids, siRNA, DNA aptamers, peptides, antibodies, non-antibody proteins, cytokines, chemokines, and chemo-attractants. Then the agent can be used to increase the ability or potency of the culture-expanded canine progenitor cells (i.e., cMAPCs) to achieve the assessed effect(s). Assessment could be in vivo or in vitro. In one example, a HUVEC tube-formation assay is used to screen for an agent that modulates the ability of the culture-expanded canine progenitor cells (e.g., cMAPCs) to provide angiogenesis in vivo and/or in vitro. In another example, an in vitro proliferation assay (e.g., MLR) is used to screen for an agent that modulates the ability of the culture-expanded canine progenitor cells (i.e., cMAPCs) to inhibit or reduce T-cell proliferation in vitro and/or in vivo.

A further use for the invention is the establishment of cell banks to provide culture-expanded canine progenitor cells (i.e., cMAPCs) for clinical administration. Cell bank construction can be done by preparing and expanding canine progenitor cells (i.e., cMAPCs) as described herein, and then storing the expanded cells from that population for future administration to a subject. Culture-expanded canine cells (i.e., cMAPCs) can be used directly from the bank or expanded prior to use.

Accordingly, the invention also is directed to diagnostic procedures conducted prior to administering these cells to a subject, the pre-diagnostic procedures including assessing the potency or ability of the cells to achieve one or more of the above effects and/or exhibit one or more of the genotypic or phenotypic markers discussed above. The culture-expanded canine progenitor cells (i.e., cMAPCs) may be taken from a cell bank and used directly or expanded prior to administration. In either case, the culture-expanded canine progenitor cells (i.e., cMAPCs) would be assessed for the potency or ability of the cells to achieve one or more of the effects and/or exhibit one or more of the genotypic or phenotypic markers.

Although the culture-expanded canine progenitor cells (i.e., cMAPCs) selected for effectiveness are necessarily assayed during the selection procedure, it may be preferable and prudent to again assay the cells prior to administration to a subject for treatment to ensure that the cells still are effective at desired levels. This is particularly preferable where the culture-expanded canine progenitor cells (i.e., cMAPCs) have been stored for any length of time, such as in a cell bank, where cells are most likely frozen during storage.

With respect to methods of treatment with the culture-expanded canine progenitor cells (i.e., cMAPCs), between the original isolation of the cells and the administration to a subject, there may be multiple (i.e., sequential) assays to ensure that the cells can still achieve the effect(s) and/or exhibit one or more of genotypic or phenotypic markers, at desired levels, after manipulations that occur within this time frame. For example, an assay may be performed after each expansion of the canine progenitor cells (i.e., cMAPCs). If culture-expanded canine progenitor cells (i.e., cMAPCs) are stored in a cell bank, they may be assayed after being released from storage. If they are frozen, they may be assayed after thawing. If the culture-expanded canine progenitor cells (i.e., cMAPCs) from a cell bank are expanded one or more subsequent times, they may be assayed after (each) expansion. Preferably, a portion of the final cell product (that is physically administered to the subject) may be assayed.

In another embodiment, culture-expanded canine progenitor cells (i.e., cMAPCs) of the present invention can be provided in kits, with appropriate packaging material. For example, a kit can comprise the following separately packaged components: culture-expanded canine progenitor cells (i.e., cMAPCs); a culture media or culture medium; and instructions for culturing the culture-expanded canine progenitor cells (i.e., cMAPCs). In some instances, the culture-expanded canine progenitor cells (i.e., cMAPCs) can be provided as frozen stocks, accompanied by separately packaged appropriate factors and media, as described herein, for culture.

Kits containing effective amounts of appropriate factors for isolation and culture of canine progenitor cells are also provided by the present invention. Upon obtaining a bone marrow aspirate from a canine, for example, a technician only need select the canine progenitor cells, using the methods described herein, with appropriate reagents provided in the kit, then culture the cells as described by the method of the present invention, using culture medium supplied as a kit component. The composition of the culture medium is described herein.

All patents and scientific references cited herein are incorporated by reference for their teachings.

The following example is for the purpose of illustration only and is not intended to limit the scope of the claims, which are appended hereto.

### EXAMPLE

Experiments were conducted to characterize the novel canine progenitor cells of the present application, which are referred to below as “canine multi-potent adult progenitor cells” or “cMAPCs”.

cMAPC Isolation

Bone marrow aspirate was obtained from the femur or tibia of a young donor dog under informed consent. The bone marrow aspirate was drawn into a syringe using a Jamshidi needle. The mononuclear fraction was isolated by Histopaque density centrifugation. Total cell count was determined (NC-200, Chemometec) and cells were plated at a density of 100,000 to 250,000 cells/cm² in a protein-coated flask. The culture medium consisted of following components: 40% MCDB-201 medium (Sigma), 35-50% αMEM medium (Lonza), 2 mM Ultraglutamine (Lonza), 10-18% FBS (Gibco), 1×ITS (insulin-transferrin-selenium, Lonza), 0.5×LA-BSA (linoleic acid-bovine serum albumin, Sigma), 100 μM L-Ascorbic acid-2-phosphate (Sigma), 10 ng/ml human/canine PDGF-BB (Biotechne/KingfisherBiotech), 50 nM dexamethasone (Sigma), 10 ng/ml canine EGF (Sino Biological), 1-10 ng/ml hFGF2 (Biotechne) and 1-10 ng/ml TGFβ1 (Biotechne). Flasks were incubated in a humidified incubator at 38° C., 5% CO₂, 5% O₂ and after 3 to 5 days, clonal expansion of the cMAPC were visible. When clones reached confluency of 50-80%, cells were lifted and passaged.

cMAPC Cell Culture

Briefly, cells were washed with PBS and afterwards detached from the plates using 1× TrypLE Select (Gibco). The trypsinization reaction was stopped by adding DPBS to the flasks. The cell solution was transferred to a conical tube and centrifuged at 500×g for 5 minutes. Next, the supernatant was removed and the cell pellet resuspended in DPBS. Cell number was determined and cells were seeded at a density of 2,000 cells/cm² in protein-coated flasks. Composition of the culture medium was as described above. Cells were incubated at 38° C., 5% CO₂, 5% O₂ and passaged every 2 to 3 days.

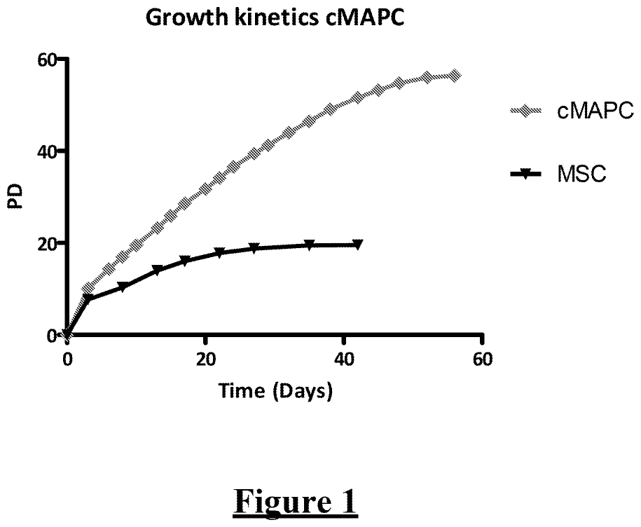

Because cells are counted at every passage, a growth curve showing the number of population doublings can be generated using the following equation: PDₕ=PDᵢ+Log₂(Cₕ/Cᵢ), where PDₕ is the population doubling at harvest, PDᵢ the initial population doubling at seeding, Cₕ the cell number at harvest, and Cᵢ the initial cell number that was seeded. When expanding the cells until senescence, cMAPC were able to go beyond 40 population doublings while canine mesenchymal stem cells (cMSC) already started to senesce at 20 population doublings (FIG. 1). Tables 1A and 1B show the doubling rates/times for cMAPCs and cMSCs (respectively), which is the number of hours necessary for one population doubling, and that for each new passage.

TABLE 1A
Doubling rates/times for cMAPCs

| # culture days | PD | Doubling time (h) |
|---|---|---|
| 3 | 10.1 | |
| 6 | 14.3 | 17.3 |
| 8 | 17.0 | 17.2 |
| 10 | 19.5 | 17.8 |
| 13 | 23.2 | 18.3 |
| 15 | 25.9 | 18.2 |
| 17 | 28.6 | 18.2 |
| 20 | 31.7 | 18.9 |
| 22 | 34.1 | 19.0 |
| 24 | 36.5 | 19.1 |
| 27 | 39.4 | 19.6 |
| 29 | 41.2 | 20.1 |
| 32 | 43.9 | 20.6 |
| 35 | 46.5 | 21.1 |
| 38 | 49.0 | 21.6 |
| 42 | 51.6 | 22.5 |
| 45 | 53.2 | 23.4 |
| 48 | 54.7 | 24.2 |
| 52 | 55.9 | 25.7 |
| 56 | 56.4 | 27.5 |

TABLE 1B
Doubling rates/times for cMSCs

| # culture days | PD | Doubling time (h) |
|---|---|---|
| 0 | 0.0 | |
| 3 | 7.7 | 9.4 |
| 8 | 10.3 | 46.0 |
| 13 | 14.0 | 32.6 |
| 17 | 16.0 | 47.8 |
| 22 | 17.8 | 67.0 |
| 27 | 18.8 | 120.0 |
| 35 | 19.5 | 282.4 |

Canine Mesenchymal Stem Cell (cMSC) Isolation

Mononuclear fractions from a fresh bone marrow aspirate were isolated using Ficoll density centrifugation. Isolated mononuclear cells were plated at a density of 200,000-350,000 cells/cm² on tissue culture treated plastic. After 3 to 5 days, clonal expansion of cMSCs was visible. Cells were lifted and passaged when clones reached 50-80% confluency.

cMSC Culture

T75 tissue culture treated flasks were used for cMSC expansion. After removing medium from the flasks, each flask was rinsed with 5 ml PBS. Four (4) ml 1× Tryple Select was then added to each flask. Flasks were incubated 2-5 minutes at room temperature. If the cells were not all detached, flasks were gently tapped. Five (5) ml of DPBS was then added to each flask and the contents thereof transferred to a conical tube. Tubes were centrifuged at 500×g for 5 minutes. Supernatant from each tube was resuspend and the cells counted. Cells were then seeded at a density of 5,000 cells/cm² in T75 culture flasks in 10 ml of a commercially available MSC medium (e.g., Lonza). Flasks were incubated at 38° C., 5.5% CO₂, and 20% O₂.

### Flow Cytometry

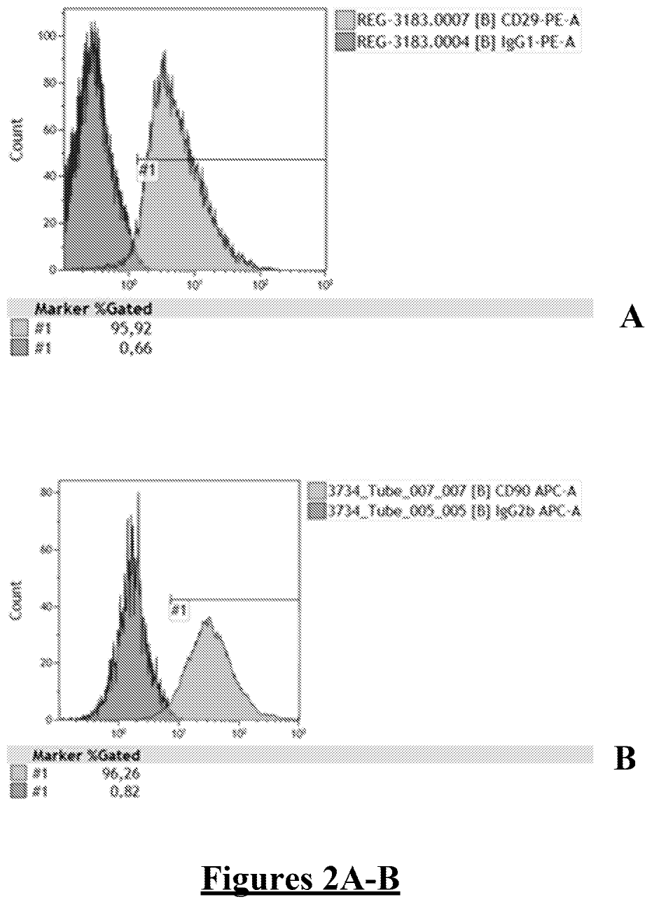

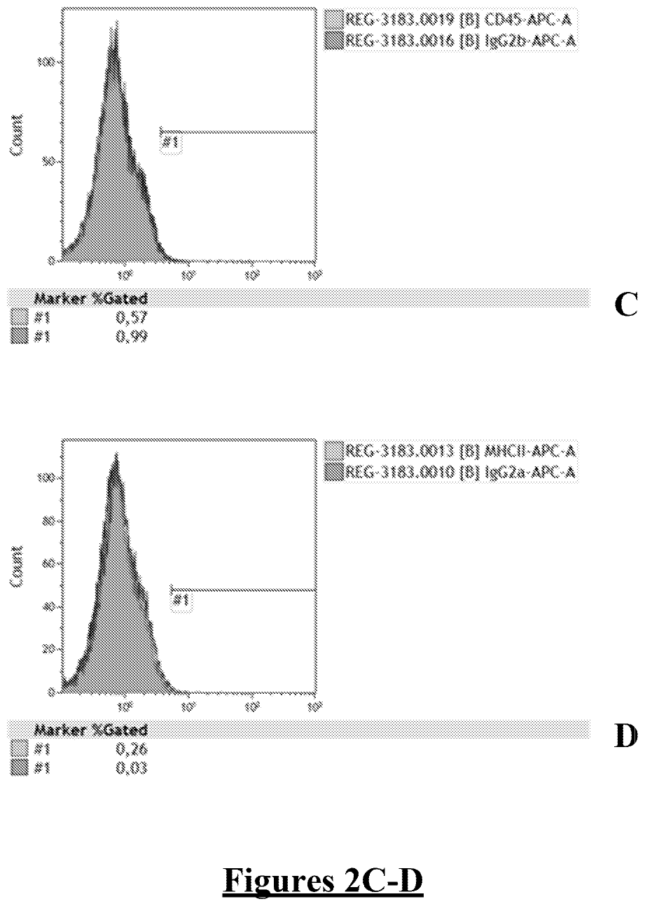

Immunophenotypic analysis of cMAPC shows that the cells express CD29 (Biolegend) and CD90 (eBioscience) and are negative for CD45 (Serotec) and HLA class II (eBioscience) (FIGS. 2A-D). For an isotype control, non-specific IgG (Becton Dickinson or Bio-Rad) was substituted for the primary antibody. cMAPC were diluted to a concentration of 1E+06 cells/ml in FACS buffer (PBS+2% BSA). 100 μl cell sample was used per staining. After adding the antibody, the cells were incubated for 30 minutes on ice and in the dark. Cells were then washed by adding 2 ml FACS buffer and centrifuged at 1000×g for 5 minutes. Supernatant was decanted and the cells were resuspended. Cells were measured on a FACS Celesta (Becton Dickinson).

### Marker Analysis by PCR

Total RNA is isolated from 100,000 to 500,000 cells using the HighPure RNA isolation kit (Roche) according to the manufacturer's instructions. The RNA concentration is measured using a Nanodrop and 250 to 500 ng RNA is used as template for the synthesis of the cDNA using the Transcriptor First Strand cDNA Synthesis kit (Roche) according to the manufacturer's instructions. The obtained cDNA is diluted 5 to 10 times.

The primers used for the transcription of a selected set of genes are shown in Table 2 below.

TABLE 2
Primer sequences for pluripotency genes

| gene | primer | sequence | length |
|---|---|---|---|
| RPL8 | F | CCATGAATCCTGTGGAGC | 64 bp |
| | R | GTAGAGGGTTTGCCGATG | |
| OCT4 | F | GTGAAGCCGGACAAGGAGAA | 179 bp |
| | R | TGGCTGAACACCTTCCCAAA | |
| NANOG | F | GAATAACCCGAATTGGAGCAG | 141 bp |
| | R | AGCGATTCCTCTTCACAGTTG | |
| SOX2 | F | AGTCTCCAAGCGACGAAAAA | 142 bp |
| | R | GCAAGAAGCCTCTCCTTGAA | |

Ribosomal Protein L8 (RPL8) was used as reference gene. GeNorm analysis has shown that this gene has a stable expression over all cMAPC and cMSC conditions. RPL8 is used to confirm quality of the cDNA.

For CD marker transcription, 5 μl of diluted cDNA was taken to perform a qPCR reaction using the LightCycler 480 SYBRGreen 1 kit (Roche) according to the manufacturer's instructions. Primer sequences for the selected surface markers are shown in Table 3.

TABLE 3
Primer sequences for surface markers
(HKG = housekeeping genes)

| | primer | sequence |
|---|---|---|
| HKG | | |
| RPL8 | F | CCATGAATCCTGTGGAGC |
| | R | GTAGAGGGTTTGCCGATG |
| RPS5 | F | TCACTGGTGAGAACCCCCT |
| | R | CCTGATTCACACGGCGTAG |
| HPRT | F | AGCTTGCTGGTGAAAAGGAC |
| | R | TTATAGTCAAGGGCATATCC |
| SDHA | F | GCCTTGGATCTCTTGATGGA |
| | R | TTCTTGGCTCTTATGCGATG |
| CDmarkers | | |
| CD13 | F | AGGGTGTGGCATCCTACCTT |
| | R | GGTCCATGATGGCGTTCACT |
| CD44 | F | TTCCAACTGCATACCCGACC |
| | R | TCGTGGTCTTTGGTAATGGGG |
| CD49c | F | ACTCTGGCCTACACACTGGA |
| | R | TGACGATGGGACGGAGTTTG |
| CD73 | F | CTCCAACACATTCCTTTACAC |
| | R | ACTCAACCTTCAAATAGCCT |
| CD90 | F | TGCTGACAGTCTTGCAGGTAG |
| | R | TACCCCCACAGTGCCATAGA |
| CD105 | F | CATCCTTCACCACCAAGAG |
| | R | CAGATTGCAGAAGGACGG |
| CD34 | F | ACCCCAAGTACCATCAAGGGAG |
| | R | TCACACAGGACTTGGGTCAG |
| CD45 | F | CCCCAGAACCTAGGCAAACAT |
| | R | ACATTGGTCACAATTCACGGTA |

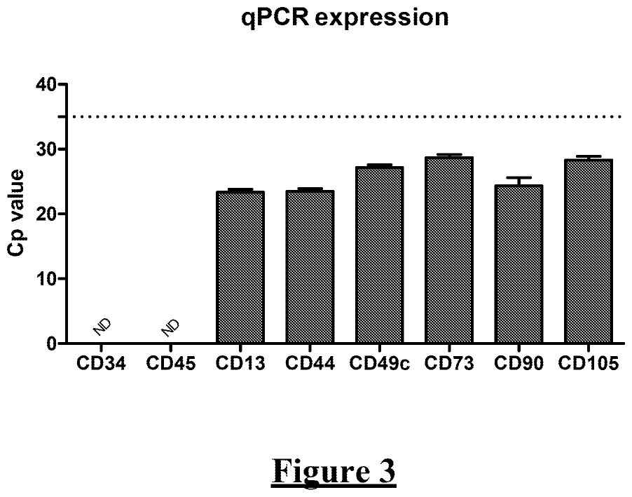

The temperature program consists of 45 cycles of 10 seconds at 95° C., 10 seconds at 60° C. and 10 seconds at 72° C. The level of mRNA expression is based on the Cq (quantification cycles) values. Markers with a Cq values below 35 are determined as being positive, above 35 as negative. A melting curve analysis was performed to test for primer-dimer formation and amplicon specificity. Results are shown in FIG. 3, where the red dotted line represents the lower limit of detection, CD34 and CD45 were not detectable, and CD13, CD44, CD49c, CD73, CD90, and CD105 showed good expression.

Markers specific for cMAPC and cMSC were tested by semiquantitative PCR. Five μl diluted cDNA was used to perform PCR reactions using iTaq DNA polymerase kit (BioRad) according to manufacturer's instructions. The following program was used: initial denaturation of 2 minutes at 95° C. followed by 28-32 cycles of amplification (15 seconds at 95° C., 15 seconds at 60° C., 30 seconds at 72° C.); and a final extension step of 5 minutes at 72° C.

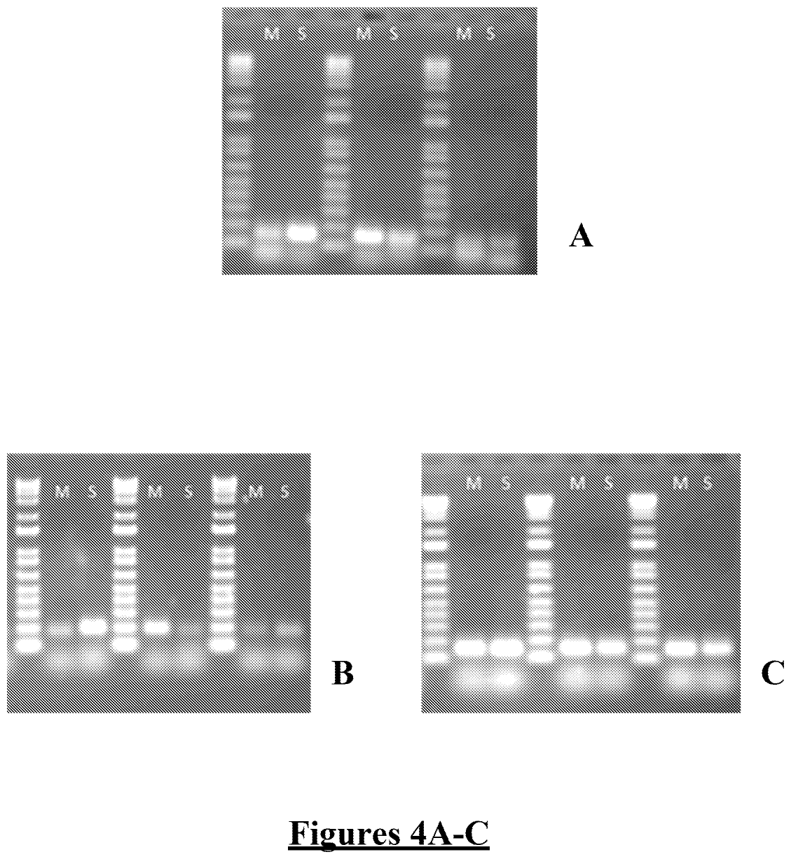

Transcription of pluripotency genes was tested by using 5 μl of diluted cDNA in a PCR reaction (iTaq DNA polymerase kit (BioRad)) using following program: initial denaturation of 2 minutes at 95° C. followed by 40-45 cycles of amplification (15 seconds at 95° C., 15 seconds at 60° C., 30 seconds at 72° C.); and a final extension step of 5 minutes at 72° C. The obtained PCR products of the cMAPC markers, the cMSC markers, and the pluripotency markers were separated on a 2% agarose gel (Invitrogen) to evaluate the presence and specificity of the products (NANOG (FIG. 4A), Oct4 (FIG. 4B), SOX2 (FIG. 4C)). Bands were stained by soaking the gel in water containing GelRed Nuclear Acid Gel Stain (Biotium). Pictures of the gel were made with BioRad Chemidoc XRS.

### Telomerase Activity

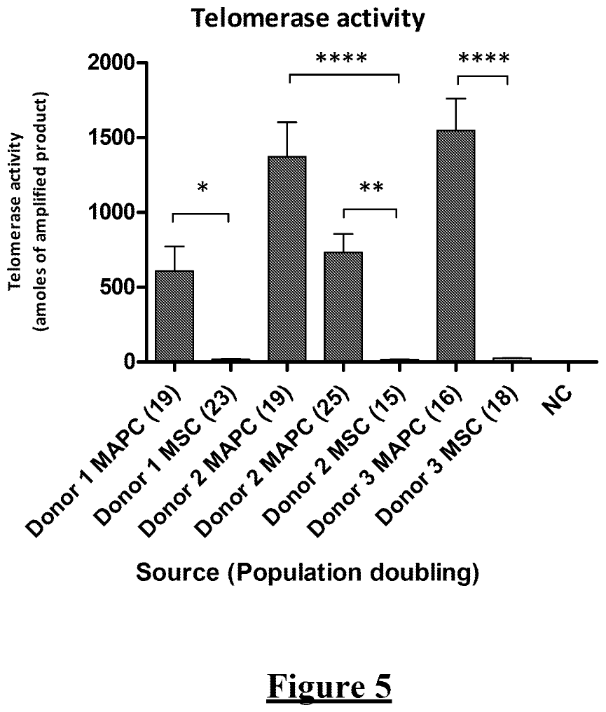

Telomerase activity of cMAPCs was determined using the TRAPeze RT Telomerase detection kit (Merck). Briefly, 1,000,000 cells were lysed with 100 μl CHAPS buffer and incubated for 30 minutes on ice. The sample was centrifuged at 12,000×g to remove any cell fragments. A 2 μl sample was added to the reaction mix. The mix was incubated for 90 minutes at 37° C. During this step, the active telomerase from the sample started to elongate a telomere template. Afterwards, the telomerase was inactivated by incubating the sample for 5 minutes at 95° C. The generated telomeres were finally quantified by realtime qPCR (FIG. 5). Significantly, cMAPCs demonstrated telomerase activity beyond 25 population doublings; whereas, cMSCs did not demonstrate telomerase activity beyond 23 population doublings.

### Adipogenic Differentiation

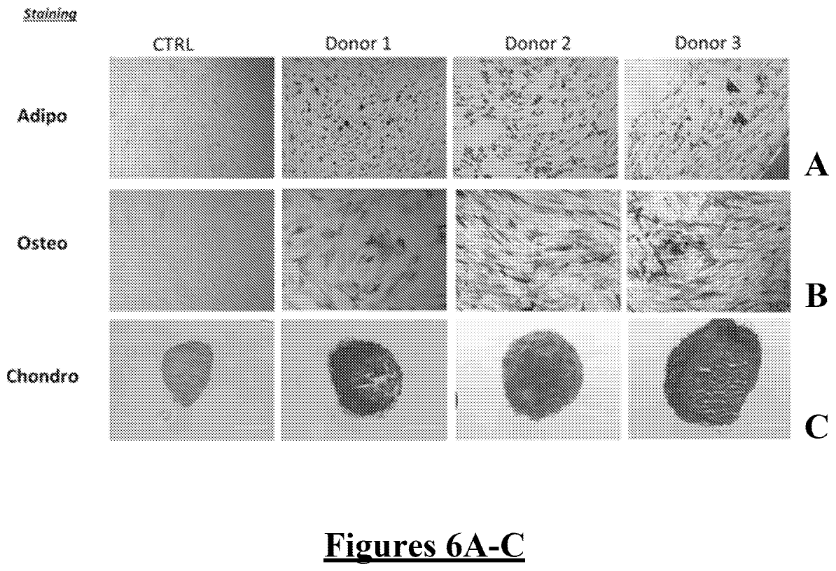

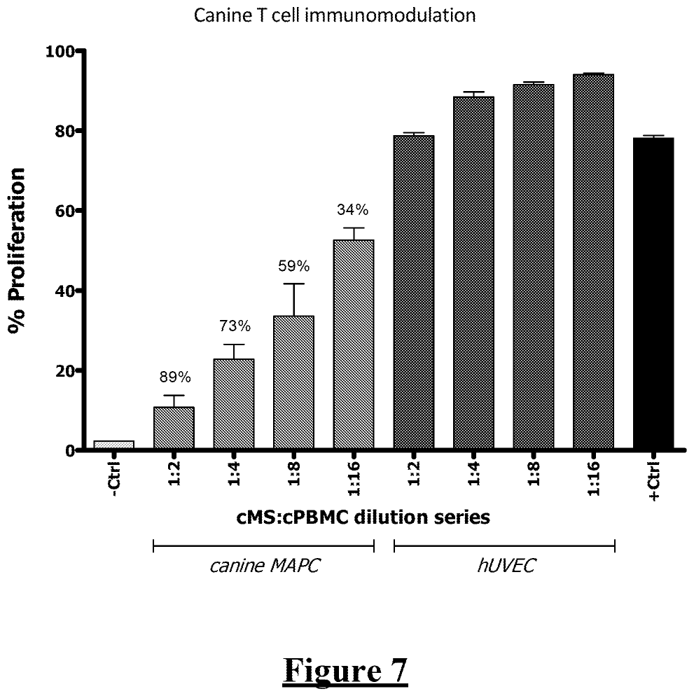

Cells were seeded at a density of 40,000 cells/cm² in control medium for 24 hours. Adipogenic control medium consisted of DMEM high glucose (4.5 g/l; Lonza), 1% L-glutamine (Lonza) and 3% FBS. Afterwards, the medium was changed to adipogenic differentiation medium (adipogenic control medium supplemented with 1 μM dexamethasone, 0.5 M 3-Isobutyl-1-methylxanthine (IBMX; Sigma), 2 μM insulin from bovine pancreas (Sigma), 33 μM biotin (Sigma), 17 μM panthothenate (Sigma), 5 μM rosiglitazone (Sigma) and 5% rabbit serum (Thermo Scientific)). Medium was refreshed twice a week and cells were collected after 9 days. Oil Red O (ORO) staining was performed to visualize lipid droplets. Cells were fixed with a citrate-buffered acetone solution (Sigma) for 30 seconds and stained with ORO (Sigma) for 10 minutes at 37° C. (FIG. 6A).

### Osteogenic Differentiation

Cells were seeded at a density of 40,000 cells/cm² in control medium (DMEM high glucose, 1% L-glutamine and 5% FBS) for 24 hours. Afterwards, the medium was changed to osteogenic differentiation medium, consisting of osteogenic control medium supplemented with 50 μM L-ascorbic acid-2P, 50 nM dexamethasone and 10 mM β-glycerophosphate (Sigma). Medium was refreshed twice a week and cells were collected after 7 days for alkaline phosphatase (ALP) staining. For the staining, cells were fixed using a citrate-buffered acetone solution (Sigma) and stained with an alkaline dye mixture (Sigma) for 10 minutes at 37° C., protected from light (FIG. 6B).

### Chondrogenic Differentiation

Cells were seeded at a density of 300,000 cells per 15 ml conical tube and centrifuged at 350×g for 5 minutes in a total volume of 1 ml chondrogenic control or differentiation medium. Chondrogenic control medium consisted of DMEM high glucose with 10% FBS. Chondrogenic basal medium consisted of DMEM high glucose, 0.625×ITS, 100 nM dexamethasone, 125 μM L-ascorbic acid-2P, 2 mM L-glutamine, 1.25×LA-BSA, 400 μg/ml Proline (Sigma) and 1 mg/ml sodium-pyruvate (Sigma). The following chondrogenic inducers were added to this medium: 10 ng/ml transforming growth factor β1 (TGF-β1) and bone morphogenic protein 2 (BMP2; Biotechne). Medium was refreshed every 3-4 days and pellets were harvested after 24 days for Alcian Blue staining. Frozen tissue blocks were prepared from the pellets. Cryosections of 5 μm were made and mounted on glass slides. Slides were first stained with a 0.5% Alcian Blue solution (Sigma) for 30 minutes followed by a Nuclear Fast Red (Vector Labs) staining for 5 minutes (FIG. 6C).

### Immunopotency Assay

An immunopotency assay was performed in a 96 well round bottom plate. In each well, 100,000 canine PBMC (peripheral mononuclear blood cells) were added to cMAPC that were plated in a serial dilution ranging from 1:2 to 1:16. cPBMC were stimulated with 0.5 μg/ml ConA (concavaline A; Sigma) and the assay was analyzed 4 days later. Briefly, the plate was centrifuged at 1000×g for 5 minutes at room temperature. Supernatant was aspirated and cells were resuspended in DPBS-FACS buffer after which the plate was centrifuged again (1000×g, 5 minutes, RT). The DPBS-FACS buffer was removed and a mix of CD3/IgG2b antibody (Abcam) was added. The mixture was incubated in the dark for 30 minutes at 4° C. Next, the plate was centrifuged (1000×g, 5 minutes, RT), the antibody mixture was removed, and the cells washed with DPBS-FACS buffer. The plate was centrifuged, supernatant removed, and a mix of GAM (goat anti mouse)-APC antibody and 7-ADD (7-aminoactinomycin D; Becton Dickinson) was added. The mixture was incubated for 15 minutes at 4° C. in the dark. The cells were washed with DBPS-FACS buffer, centrifuged (1000×g, 5 minutes, RT), resuspended in DBPS-FACS buffer, and the plate measured using a FACS Celesta (Becton Dickinson). As indicated in FIG. 7, cMAPCs were able to substantially inhibit or reduce T-cell proliferation.

### Tube Formation (Angiogenic Potential)

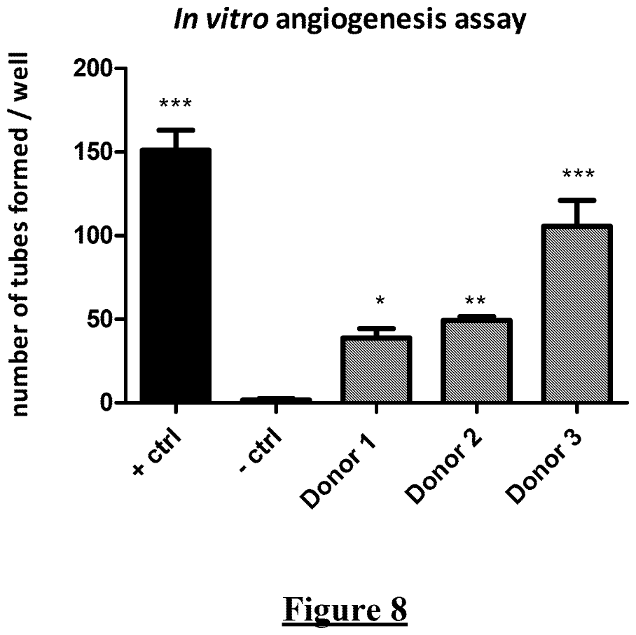

In brief, 55,000 human umbilical vein endothelial cells (hUVEC) were seeded in a 24 well plate coated with Matrigel (Corning). Conditioned medium of cMAPC was added and pictures of each well were taken after approximately 18 hours. The number of formed tubes was counted for each well. Statistical analysis between conditions was performed using one-way ANOVA. Conditioned medium from cMAPC was able to induce tube formation (FIG. 8).

### Cytogenic Analysis

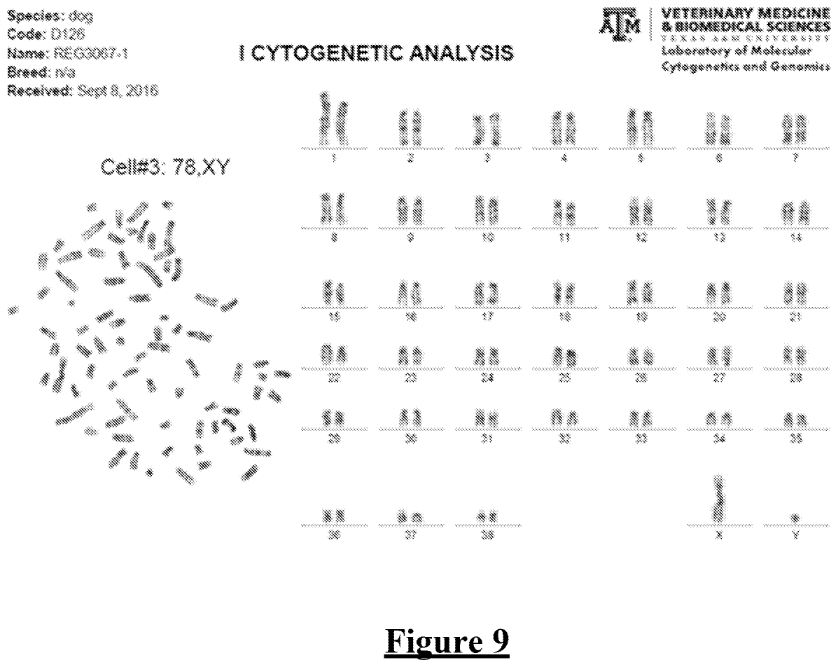

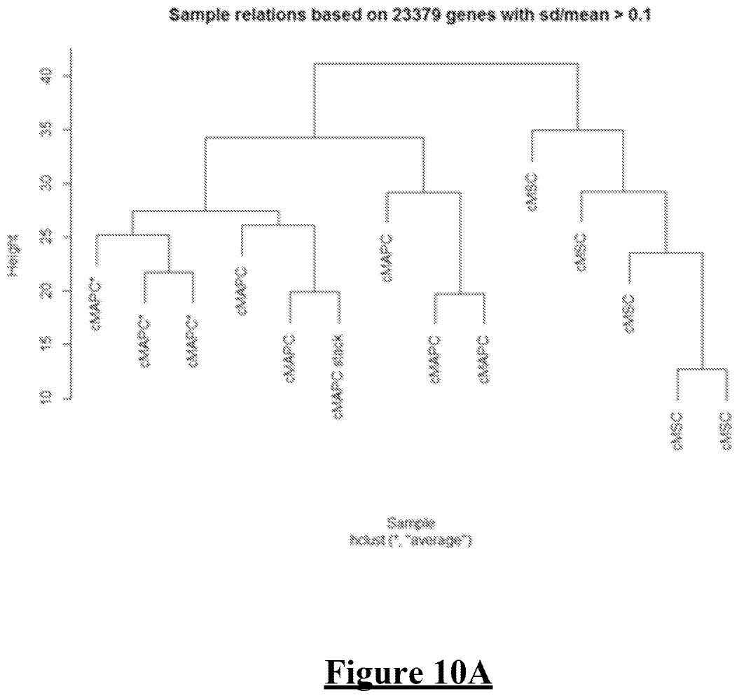

Cells were prepared for cytogenetic analysis by means of G-Banding. In brief, cMAPC were subjected to a colcemid treatment by adding demecolcine (0.1 μg/ml; Sigma) and etidiumbromide (10 μg/ml; Sigma) to the medium followed by an incubation step of 1 hour at 37° C. Thereafter, cells were subjected to a hypotonic treatment by adding hypotonic solution (Rainbow Scientific) to the medium and incubating for 40 minutes at 37° C. Next, cells were scraped off and transferred to a 15 ml conical tube. Cells were fixed by adding 500 μl fixative (methanol: acetic acid; 3:1) per 10 ml of supernatant. The solution was centrifuged for 10 minutes at 500×g after which the supernatant was removed, the cell pellet mixed, and ice cold methanol: acetic acid fixative added dropwise up to 15 ml. The cells were mixed gently but thoroughly in the fixative, and the fixing step was repeated 3 times. One ml of the cell suspension (after removal of the supernatant) was transferred to a microcentrifuge tube, which was then filled up with fixative and sent to an external lab for karyotypic analysis by means of G-banding (FIG. 9).

### Microarray

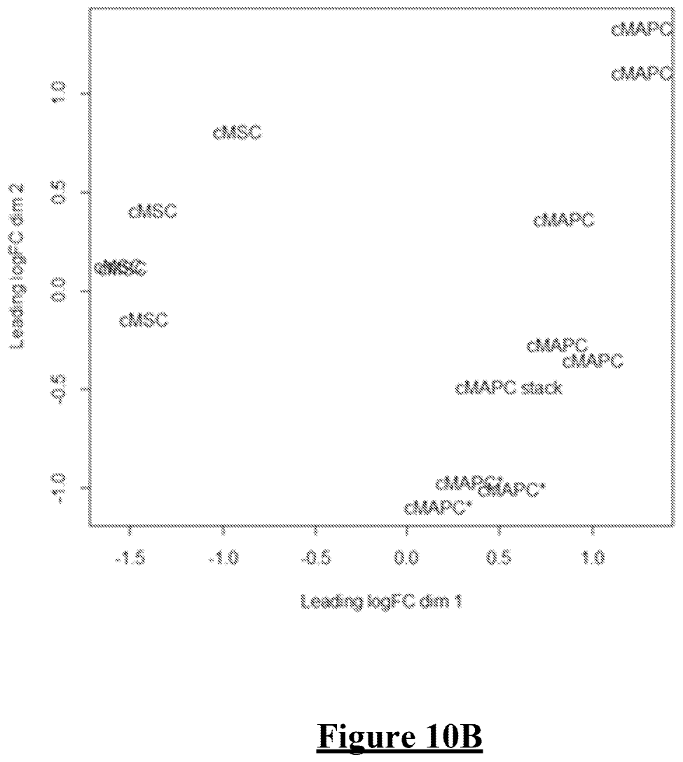

RNA was extracted from frozen cell pellets and quality control and quantification of the corresponding RNA for each sample was performed on the Agilent BioAnalyzer. The RNA was run on the Affymetrix Canine Genome 2.0 arrays (Affymetrix). R statistical language with Oligo and Limma packages (Bioconductor) were used for RMA normalization and assessment of differential expression between the different conditions. Differential expression was calculated based on moderated t statistics with a Bayesian adjusted denominator. Dendogram (FIG. 10A) and PCA (principal component analysis) plot (FIG. 10B) were used to show that cMAPC and cMSC form two separate clusters can be considered as two distinct cell populations based on total gene expression.

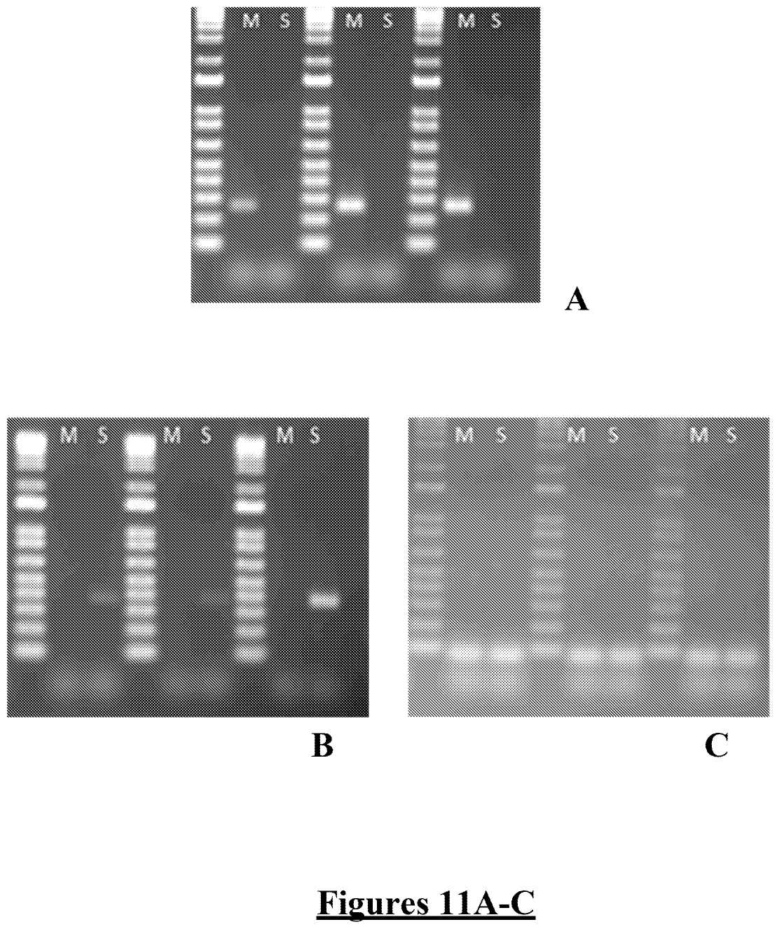

In a separate microarray experiment to confirm cMAPC and cMSC specific markers, identified from the microarray, RNA was extracted from cMAPC (M) and cMSC(S) from 3 different donors. The RNA was converted into cDNA. The cDNA was used to perform PCR for genes IL1R2 (FIG. 11A) and NOV (FIG. 11B). Primer sequences for the marker genes are shown in Table 4.

TABLE 4
Primer sequences for IL1R2, NOV and RPL8

| gene | primer | sequence | length |
|---|---|---|---|
| RPL8 | F | CCATGAATCCTGTGGAGC | 64 bp |
| | R | GTAGAGGGTTTGCCGATG | |
| IL1R2 | F | CCCAGGAGGACGGGGTATAA | 262 bp |
| | R | AGTTTTCTGCAGCCCCTGG | |
| NOV | F | GCATCTGCATGGCGGTAGA | 366 bp |
| | R | TGCCACAGCTCTTGGAACAT | |

The PCR products were then loaded on a 2% agarose gel to visualize the expression of the genes. It can be concluded that cMAPC show expression of the gene IL1R2, while this expression is absent in cMSC. Expression of NOV is absent in cMAPC and present in cMSC. Ribosomal Protein L8 (RPL8) (FIG. 11C) was used as reference gene. GeNorm analysis showed that this gene has a stable expression over all MAPC and MSC conditions. The gel staining for RPL8 showed that equal amounts of cDNA were loaded onto the gel for the different conditions.

1. Culture-expanded canine progenitor cells that have a population doubling rate of less than about 24 hours, a normal karyotype, can differentiate into at least two cell types of the mesodermal germ layer, and are post-natal somatic cells.

2. The culture-expanded canine progenitor cells of claim 1, that have a population doubling rate of less than about 24 hours, a normal karyotype, express telomerase, and are post-natal somatic cells.

3. The culture-expanded canine progenitor cells of claim 1, that have a population doubling rate of less than about 24 hours, a normal karyotype, express oct-4, and are post-natal somatic cells.

4. The culture-expanded canine progenitor cells of claim 1, that have a population doubling rate of less than about 24 hours, a normal karyotype, have undergone at least 40 population doublings in culture, and are post-natal somatic cells.

5. The culture-expanded canine progenitor cells of claim 1, wherein the cells have undergone at least 40 population doublings in culture.

6. (canceled)

7. (canceled)

8. (canceled)

9. The culture-expanded canine progenitor cells of claim 1 being derived from bone marrow, adipose tissue, umbilical cord blood, or placental tissue.

10. The culture-expanded canine progenitor cells of claim 1 being positive for one of more of:
expression of CD90 and negative for expression of CD45 and CD34;
expression of CD29;
expression of one or more of PTHLH, CD13, CD44, CD49c, CD73, CD105, and IL1R2;
expression of IL1R2; or
expression of nanog, sox-2 and oct-4.

11. (canceled)

12. The culture-expanded canine progenitor cells of claim 1 being negative for one or more of:
expression of MHC Class II; or
expression of rex-1 and NOV.

13. (canceled)

14. (canceled)

15. (canceled)

16. (canceled)

17. The culture-expanded canine progenitor cells of claim 1 expressing one or more of:
telomerase up to about 55 population doublings in culture; or
oct-4 up to about 55 population doublings in culture.

18. (canceled)

19. The culture-expanded canine progenitor cells of claim 1, wherein the cells are capable of at least one of:
differentiating into at least two cell types of the mesodermal germ layer up to about 55 population doublings in culture;
reducing or inhibiting T-cell proliferation in vitro and/or in vivo;
providing angiogenesis in vitro and/or in vivo;
differentiating into at least two cell types of the mesodermal germ layer; or
differentiating into at least two of an osteoblast, an adipocyte, and a chondrocyte.

20. (canceled)

21. (canceled)

22. (canceled)

23. (canceled)

24. The culture-expanded canine progenitor cells of claim 1, wherein the cells are not tumorigenic, do not form teratomas, are not transformed, and are not immortalized.

25. (canceled)

26. (canceled)

27. A pharmaceutical composition comprising the culture-expanded progenitor cells of claim 1 and a pharmaceutically acceptable carrier.

28. (canceled)

29. (canceled)

30. (canceled)

31. A method for preparing the culture-expanded canine progenitor cells of claim 1, the method comprising:
obtaining tissue from a canine; establishing a population of adherent cells;
selecting cells that positively express CD90 and/or do not express at least one of CD45 and CD34; and
expanding the selected cells in a culture medium.

32. A method to construct a cell bank, the method comprising expanding and storing the culture-expanded progenitor cells of claim 1 for future administration to a subject.

33. A method for drug discovery, the method comprising exposing the culture-expanded progenitor cells of claim 1 to an agent to assess one or more effects of the agent on the cells.

34. A method for treating an inflammatory condition in a canine comprising administering to the canine a therapeutically effective amount of the canine progenitor cells of claim 1.

35. The method of claim 34 wherein the inflammatory condition is a chronic inflammatory condition or an acute inflammatory condition.

36. The method of claim 35 wherein the acute or chronic inflammatory condition is one of dermatitis, inflammatory eye disease, inflammatory brain disease, inflammatory airway disease, and inflammatory bowel disease.

37. The method of claim 36 wherein the dermatitis is atopic dermatitis.

38. The method of claim 36 wherein the inflammatory eye disease is keratoconjunctivitis.

39. The method of claim 36 wherein the inflammatory brain disease is meningoencephalomyelitis.

40. The method of claim 34 wherein the inflammatory condition is an autoimmune disease.

41. A method for treating a musculoskeletal disorder in a canine comprising administering to the canine a therapeutically effective amount of the canine progenitor cells of claim 1.

42. The method of claim 41 wherein the musculoskeletal disorder is one of osteoarthritis and cruciate ligament rupture.

43. The method of claim 42 wherein the cruciate ligament rupture is a partial cruciate ligament rupture.

44. The method of claim 41 wherein the musculoskeletal disorder is a spinal condition.

45. The method of claim 44 wherein the spinal condition is one of a spinal cord injury and intervertebral disc disease.
